SEQUENCE LISTING (23 sequences)

SEQ ID NO: 1 - protein, 986 aa, Artificial Sequence
  Synthetic polypeptide
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 3027 bp, Artificial Sequence
  Synthetic polynucleotide
atggacatgagggtgcccgctcagctcctggggctcctgctgctgtggctgagaggtgcgcgctgtcaggtgcagctcgtggaatccggtggcggcttggtgaaacctggcgagtcactgagattgtcatgcgctgcatcaggttttacattcagtgattactatatgtactgggtacggcaggcacctggaaaatgccttgagtgggtcgctattatcagtgacggcgggtattatacctattactccgatatcattaaaggaaggtttaccatcagccgagataacgccaaaaactccctttaccttcagatgaacagtcttaaagctgaggacactgctgtttactattgtgctagagggttccccctgctgcgtcacggcgcaatggattactggggccagggcacccttgtaaccgtaagcagtggtggggggggtagtggtggtggtggctctggcggtggaggatcagacatccagatgacccaaagcccctctagtctcagcgcaagtgtaggcgaccgggtaaccattacatgtaaggcatcacagaatgtggacacaaatgttgcttggtatcagcagaagccaggacaggctcctaagtcacttatctacagtgcaagttatgtgtattgggacgttccttcccgcttttctggttccgctagtggaactgacttcactttgactatttcatcagttcagtctgaggacttcgccacatattactgccaacaatatgatcagcagctcattacctttggctgtggcacaaagctggaaatcaagtccggaggtggtggatccgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcatcagaagtaaatataataattatgcaacatattatgccgattcagtgaaagacaggttcaccatctccagagatgattcaaaaaacactgcctatctacaaatgaacaacttgaagactgaggacactgccgtgtactactgtgtgagacatgggaacttcggtaatagctacatctcctactgggcttactggggccaagggactctggtcaccgtgtcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaatcggtgggactaagttcctcgcccccggtactcctgccagattctcaggctccctgcttggaggcaaggctgccctcaccctctcaggggtacagccagaggatgaggcggaatattactgtgttctatggtacagcaaccgctgggtgttcggtggaggaaccaaactgactgtcctaggcggtggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggtggaggaggtagcggaggaggcggctccggagggggcggatctggcggcggaggaagcggtgggggcggctccggagggggcggctctgacaagacccacacatgtcctccgtgccccgcacccgagctgttgggaggaccctcggtgtttttgtttcctcccaagccaaaggacacgttgatgatttcgcgcactccagaggtcacgtgtgtagtcgtggacgtgtcacatgaggaccccgaggtaaagttcaattggtatgtggacggagtcgaggtccataacgccaaaacaaagccgtgcgaagaacagtacgggtcaacgtataggtgcgtcagcgtcctcactgtgctgcaccaagactggctcaatggtaaagaatacaagtgcaaggtgtcgaacaaggccctccctgcccctatcgagaaaaccatctccaaagcgaaggggcagccgcgagaaccccaagtctacacgctgccgccctcgcgggaggaaatgaccaaaaaccaggtgtcgcttacgtgtcttgtgaaagggttctatccatcagatatcgcggtcgagtgggagtcgaacggccagcccgaaaacaattacaaaacaacacctccggtcctcgattcagatggaagcttcttcttgtattcgaagctgaccgtcgataagtcaaggtggcaacagggaaatgtgttctcgtgctcagtgatgcacgaggctctgcataaccactatacgcagaaatcattgtcgctcagccccggtaaatag

SEQ ID NO: 3 - DNA, 2961 bp, Artificial Sequence
  Synthetic polynucleotide
caggtgcagctcgtggaatccggtggcggcttggtgaaacctggcgagtcactgagattgtcatgcgctgcatcaggttttacattcagtgattactatatgtactgggtacggcaggcacctggaaaatgccttgagtgggtcgctattatcagtgacggcgggtattatacctattactccgatatcattaaaggaaggtttaccatcagccgagataacgccaaaaactccctttaccttcagatgaacagtcttaaagctgaggacactgctgtttactattgtgctagagggttccccctgctgcgtcacggcgcaatggattactggggccagggcacccttgtaaccgtaagcagtggtggggggggtagtggtggtggtggctctggcggtggaggatcagacatccagatgacccaaagcccctctagtctcagcgcaagtgtaggcgaccgggtaaccattacatgtaaggcatcacagaatgtggacacaaatgttgcttggtatcagcagaagccaggacaggctcctaagtcacttatctacagtgcaagttatgtgtattgggacgttccttcccgcttttctggttccgctagtggaactgacttcactttgactatttcatcagttcagtctgaggacttcgccacatattactgccaacaatatgatcagcagctcattacctttggctgtggcacaaagctggaaatcaagtccggaggtggtggatccgaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcatcagaagtaaatataataattatgcaacatattatgccgattcagtgaaagacaggttcaccatctccagagatgattcaaaaaacactgcctatctacaaatgaacaacttgaagactgaggacactgccgtgtactactgtgtgagacatgggaacttcggtaatagctacatctcctactgggcttactggggccaagggactctggtcaccgtgtcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaatcggtgggactaagttcctcgcccccggtactcctgccagattctcaggctccctgcttggaggcaaggctgccctcaccctctcaggggtacagccagaggatgaggcggaatattactgtgttctatggtacagcaaccgctgggtgttcggtggaggaaccaaactgactgtcctaggcggtggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgtgcgaggagcagtacggcagcacgtaccgttgcgtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggtggaggaggtagcggaggaggcggctccggagggggcggatctggcggcggaggaagcggtgggggcggctccggagggggcggctctgacaagacccacacatgtcctccgtgccccgcacccgagctgttgggaggaccctcggtgtttttgtttcctcccaagccaaaggacacgttgatgatttcgcgcactccagaggtcacgtgtgtagtcgtggacgtgtcacatgaggaccccgaggtaaagttcaattggtatgtggacggagtcgaggtccataacgccaaaacaaagccgtgcgaagaacagtacgggtcaacgtataggtgcgtcagcgtcctcactgtgctgcaccaagactggctcaatggtaaagaatacaagtgcaaggtgtcgaacaaggccctccctgcccctatcgagaaaaccatctccaaagcgaaggggcagccgcgagaaccccaagtctacacgctgccgccctcgcgggaggaaatgaccaaaaaccaggtgtcgcttacgtgtcttgtgaaagggttctatccatcagatatcgcggtcgagtgggagtcgaacggccagcccgaaaacaattacaaaacaacacctccggtcctcgattcagatggaagcttcttcttgtattcgaagctgaccgtcgataagtcaaggtggcaacagggaaatgtgttctcgtgctcagtgatgcacgaggctctgcataaccactatacgcagaaatcattgtcgctcagccccggtaaatag

SEQ ID NO: 4 - DNA, 3027 bp, Artificial Sequence
  Synthetic polynucleotide
atggatatgcgcgtgcccgctcagctgctggggctgcttctgctctggcttcgcggggcccgctgtcaggtgcaactggtggaaagcggtggcggccttgtgaagcctggggaatccctcaggctctcctgtgctgctagtggcttcacgttcagcgactactacatgtattgggtgcggcaggctccaggaaagtgccttgagtgggtcgccatcatttccgatggcgggtattacacctactacagcgacatcatcaagggtcggtttaccattagccgcgacaatgccaagaacagcctgtatctgcagatgaacagtctgaaagccgaggatactgccgtttattactgtgcaagagggtttcccttgttgcgtcacggcgcaatggactactggggtcaaggaacactggtgacagtatcatcaggaggtggagggtctggcggcggaggtagtggaggaggtgggtccgatattcagatgactcagtctccttcctctctgagcgcttctgtaggcgatcgagtcaccataacctgcaaagcaagccagaatgtggacacaaacgtggcatggtaccaacagaagccaggccaagctcccaagtctctgatctattctgcctcatacgtctattgggatgttcccagtcggtttagtggatctgcctctgggacagacttcactctgactatctcctccgttcaatccgaggactttgccacctactattgccagcagtatgatcagcagctcatcacattcggctgtgggaccaaactggagattaaatccggaggtggagggagcgaagtgcagctcgttgagagcggcggaggccttgtgcagcctggaggatccctgaagctgagctgtgccgcatctgggttcacattcaacaagtatgccatgaattgggtgcgtcaggctcctggtaaaggcttggagtgggtagcccggattaggagcaaatataataactatgccacctactatgctgactctgtcaaggaccgctttacaatctctcgggatgactccaagaatactgcctacctccagatgaacaacctcaagacggaggataccgccgtctactattgcgtccgacatggcaactttgggaactcctacatctcctattgggcttattggggccaggggactcttgtcacagtctcatccggcggtgggggctctggtggaggtgggtcaggaggtggtgggagccaaaccgttgtaacacaggagccctccttgactgtgtcacctggcggaacggtgacgctgacttgtggctcatcaactggagccgtaacatctggaaattaccctaactgggtgcagcagaagcctggtcaggcacccagaggactgataggcggaaccaaatttctcgctccaggtactccagcccggttttccgggtccttgctgggcggtaaggctgccctgacactgagcggagttcagccagaggatgaagcagaatactattgcgtgctgtggtactccaacaggtgggtatttggaggcggcaccaagctcacggtcctcggcggaggtggagacaaaacacacacttgtcctccctgtcctgcacccgaacttctgggaggcccctctgtgttccttttcccacccaagcccaaagataccttgatgatcagccgtacgccagaggttacatgcgtggtcgttgacgtgtctcatgaagatcccgaagtcaagttcaactggtacgtggatggggtggaggtacataatgcaaagaccaagccctgtgaggagcagtatgggtcaacctatcggtgtgtgtctgtgctgaccgtgctgcatcaagattggttgaatgggaaggaatataagtgcaaggtgtcaaataaggctctgcccgcacccattgaaaagaccataagtaaggccaaggggcagcctagggaaccccaggtatacactctgcctccatcccgtgaggagatgacaaagaatcaagtgagcctcacctgtctggttaaaggcttctaccccagtgatatcgctgtggagtgggagtcaaacgggcagcccgaaaacaactacaaaaccacacctccagtcttggatagcgacggatccttctttctgtactctaaactcaccgtggacaaaagtagatggcagcaaggcaacgttttcagttgctctgtgatgcacgaagctctccataatcattatactcagaagagcctgtccctttctcctggaaaaggggggggtggctccggcggtggcggcagtggaggcggaggctctggtggtggcggttccggtggcgggggaagcggcggcggtgggagtgacaagacacatacctgtcctccatgcccagctcccgaactgttgggcggacccagcgtgtttctgtttccaccaaagcctaaagacaccctgatgatttcaaggactcctgaggtgacatgcgtggtagtggatgtcagccacgaggaccccgaagtgaaattcaattggtatgtagacggggtggaggtccacaatgccaagactaaaccctgcgaggagcagtacgggtccacttaccgatgcgttagtgttctcacagtccttcaccaggactggctgaacggaaaggagtacaaatgcaaagtgtccaacaaagccctgccagccccaatcgagaaaacaatcagcaaggccaagggtcaacctcgagaaccccaagtgtatactctgccaccctctcgcgaagagatgaccaagaatcaggtttccctcacctgtttggtcaaaggcttttacccctctgacattgctgttgaatgggagagtaacggccagcctgagaacaactataaaaccaccccaccagtgcttgatagtgatggctccttcttcctgtacagcaagctgacagtggacaaatctcgctggcaacagggcaatgtctttagttgctccgtcatgcacgaggcactgcacaatcactacactcagaagtcactgagtctgagccctgggaagtag

SEQ ID NO: 5 - DNA, 2961 bp, Artificial Sequence
  Synthetic polynucleotide
caggtgcaactggtggaaagcggtggcggccttgtgaagcctggggaatccctcaggctctcctgtgctgctagtggcttcacgttcagcgactactacatgtattgggtgcggcaggctccaggaaagtgccttgagtgggtcgccatcatttccgatggcgggtattacacctactacagcgacatcatcaagggtcggtttaccattagccgcgacaatgccaagaacagcctgtatctgcagatgaacagtctgaaagccgaggatactgccgtttattactgtgcaagagggtttcccttgttgcgtcacggcgcaatggactactggggtcaaggaacactggtgacagtatcatcaggaggtggagggtctggcggcggaggtagtggaggaggtgggtccgatattcagatgactcagtctccttcctctctgagcgcttctgtaggcgatcgagtcaccataacctgcaaagcaagccagaatgtggacacaaacgtggcatggtaccaacagaagccaggccaagctcccaagtctctgatctattctgcctcatacgtctattgggatgttcccagtcggtttagtggatctgcctctgggacagacttcactctgactatctcctccgttcaatccgaggactttgccacctactattgccagcagtatgatcagcagctcatcacattcggctgtgggaccaaactggagattaaatccggaggtggagggagcgaagtgcagctcgttgagagcggcggaggccttgtgcagcctggaggatccctgaagctgagctgtgccgcatctgggttcacattcaacaagtatgccatgaattgggtgcgtcaggctcctggtaaaggcttggagtgggtagcccggattaggagcaaatataataactatgccacctactatgctgactctgtcaaggaccgctttacaatctctcgggatgactccaagaatactgcctacctccagatgaacaacctcaagacggaggataccgccgtctactattgcgtccgacatggcaactttgggaactcctacatctcctattgggcttattggggccaggggactcttgtcacagtctcatccggcggtgggggctctggtggaggtgggtcaggaggtggtgggagccaaaccgttgtaacacaggagccctccttgactgtgtcacctggcggaacggtgacgctgacttgtggctcatcaactggagccgtaacatctggaaattaccctaactgggtgcagcagaagcctggtcaggcacccagaggactgataggcggaaccaaatttctcgctccaggtactccagcccggttttccgggtccttgctgggcggtaaggctgccctgacactgagcggagttcagccagaggatgaagcagaatactattgcgtgctgtggtactccaacaggtgggtatttggaggcggcaccaagctcacggtcctcggcggaggtggagacaaaacacacacttgtcctccctgtcctgcacccgaacttctgggaggcccctctgtgttccttttcccacccaagcccaaagataccttgatgatcagccgtacgccagaggttacatgcgtggtcgttgacgtgtctcatgaagatcccgaagtcaagttcaactggtacgtggatggggtggaggtacataatgcaaagaccaagccctgtgaggagcagtatgggtcaacctatcggtgtgtgtctgtgctgaccgtgctgcatcaagattggttgaatgggaaggaatataagtgcaaggtgtcaaataaggctctgcccgcacccattgaaaagaccataagtaaggccaaggggcagcctagggaaccccaggtatacactctgcctccatcccgtgaggagatgacaaagaatcaagtgagcctcacctgtctggttaaaggcttctaccccagtgatatcgctgtggagtgggagtcaaacgggcagcccgaaaacaactacaaaaccacacctccagtcttggatagcgacggatccttctttctgtactctaaactcaccgtggacaaaagtagatggcagcaaggcaacgttttcagttgctctgtgatgcacgaagctctccataatcattatactcagaagagcctgtccctttctcctggaaaaggggggggtggctccggcggtggcggcagtggaggcggaggctctggtggtggcggttccggtggcgggggaagcggcggcggtgggagtgacaagacacatacctgtcctccatgcccagctcccgaactgttgggcggacccagcgtgtttctgtttccaccaaagcctaaagacaccctgatgatttcaaggactcctgaggtgacatgcgtggtagtggatgtcagccacgaggaccccgaagtgaaattcaattggtatgtagacggggtggaggtccacaatgccaagactaaaccctgcgaggagcagtacgggtccacttaccgatgcgttagtgttctcacagtccttcaccaggactggctgaacggaaaggagtacaaatgcaaagtgtccaacaaagccctgccagccccaatcgagaaaacaatcagcaaggccaagggtcaacctcgagaaccccaagtgtatactctgccaccctctcgcgaagagatgaccaagaatcaggtttccctcacctgtttggtcaaaggcttttacccctctgacattgctgttgaatgggagagtaacggccagcctgagaacaactataaaaccaccccaccagtgcttgatagtgatggctccttcttcctgtacagcaagctgacagtggacaaatctcgctggcaacagggcaatgtctttagttgctccgtcatgcacgaggcactgcacaatcactacactcagaagtcactgagtctgagccctgggaagtag

SEQ ID NO: 6 - protein, 22 aa, Artificial Sequence
  Synthetic polypeptide
MDMRVPAQLLGLLLLWLRGARC

SEQ ID NO: 7 - protein, 19 aa, Artificial Sequence
  Synthetic polypeptide
MAWALLLLTLLTQGTGSWA

SEQ ID NO: 8 - protein, 19 aa, Artificial Sequence
  Synthetic polypeptide
MTCSPLLLTLLIHCTGSWA

SEQ ID NO: 9 - protein, 19 aa, Artificial Sequence
  Synthetic polypeptide
MEWTWRVLFLVAAATGAHS

SEQ ID NO: 10 - protein, 19 aa, Artificial Sequence
  Synthetic polypeptide
MEWSWVFLFFLSVTTGVHS

SEQ ID NO: 11 - protein, 22 aa, Artificial Sequence
  Synthetic polypeptide
MDIRAPTQLLGLLLLWLPGAKC

SEQ ID NO: 12 - protein, 22 aa, Artificial Sequence
  Synthetic polypeptide
MDIRAPTQLLGLLLLWLPGARC

SEQ ID NO: 13 - protein, 22 aa, Artificial Sequence
  Synthetic polypeptide
MDMRAPTQLLGLLLLWLPGARC

SEQ ID NO: 14 - protein, 22 aa, Artificial Sequence
  Synthetic polypeptide
MDTRAPTQLLGLLLLWLPGATF

SEQ ID NO: 15 - protein, 22 aa, Artificial Sequence
  Synthetic polypeptide
MDTRAPTQLLGLLLLWLPGARC

SEQ ID NO: 16 - protein, 19 aa, Artificial Sequence
  Synthetic polypeptide
METGLRWLLLVAVLKGVQC

SEQ ID NO: 17 - protein, 21 aa, Artificial Sequence
  Synthetic polypeptide
METGLRWLLLVAVLKGVQCQE

SEQ ID NO: 18 - protein, 20 aa, Artificial Sequence
  Synthetic polypeptide
MEAPAQLLFLLLLWLPDTTG

SEQ ID NO: 19 - protein, 20 aa, Artificial Sequence
  Synthetic polypeptide
METPAQLLFLLLLWLPDTTG

SEQ ID NO: 20 - DNA, 66 bp, Artificial Sequence
  Synthetic polynucleotide
atggatatgcgcgtgcccgctcagctgctggggctgcttctgctctggcttcgcggggcccgctgt

SEQ ID NO: 21 - DNA, 57 bp, Artificial Sequence
  Synthetic polynucleotide
atggcctgggctctgctgctcctcaccctcctcactcaggggacagggtcctgggcc

SEQ ID NO: 22 - DNA, 57 bp, Artificial Sequence
  Synthetic polynucleotide
atggcctgggctctgctgctcctcaccctcctcactcaggggaccgggtcctgggcc

SEQ ID NO: 23 - protein, 769 aa, Artificial Sequence
  Synthetic polypeptide
EDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0209115 A1
(43) Pub. Date: Jun. 27, 2024

(54) METHODS FOR REDUCING LOW MOLECULAR WEIGHT SPECIES OF RECOMBINANTLY-PRODUCED PROTEINS

(71) Applicants: AMGEN INC., Thousand Oaks, CA (US); AMGEN RESEARCH (MUNICH) GmbH, Munich (DE)

(72) Inventors: Anika MENTHE, Munich (DE); Joseph BASCONI, Cambridge, MA (US); Nitya M. JACOB, Oak Park, CA (US); Russell JAMPOL, Cambridge, MA (US); Huong Thi Ngoc LE, Newbury Park, CA (US); Kim LE, Castro Valley, CA (US); Jessica PEDROSO, Ashland, MA (US); Jennitte STEVENS, Thousand Oaks, CA (US); Sreekanth SURAVAJJALA, Arlington, MA (US); Charilyn TEJAMO, Moorpark, CA (US); Pheng YAM, Oak Park, CA (US)

(73) Assignees: AMGEN INC., Thousand Oaks, CA (US); AMGEN RESEARCH (MUNICH) GmbH, Munich (DE)

(21) Appl. No.: 18/556,998

(22) PCT Filed: Apr. 28, 2022
(86) PCT No.: PCT/US2022/026707

§ 371 Date: Oct. 24, 2023

Related U.S. Application Data
(60) Provisional application No. 63/181,903, filed on Apr. 29, 2021.

(51) Int. Cl. C07K 16/40; C07K 16/28; C07K 16/46

(52) U.S. Cl. CPC C07K 16/40; C07K 16/2809; C07K 16/468; C07K 2317/31; C07K 2317/622; C07K 2319/02

(57) ABSTRACT

The present invention relates to methods for reducing low molecular weight species of recombinantly-produced proteins. In particular, methods of reducing the formation of low molecular weight species produced by a host cell during the cell culture process through pH control of the production cell culture are disclosed. Also disclosed are methods for reducing or eliminating the generation of alternative splice variants by a host cell during production of a recombinant protein.

### CROSS-REFERENCE TO RELATED APPLICATIONS

This application claims the benefit of U.S. Provisional Application No. 63/181,903, filed Apr. 29, 2021, which is hereby incorporated by reference in its entirety.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Apr. 11, 2022, is named A-2734-WO01-SEC_ST25 and is 36 kilobytes in size.

### FIELD OF THE INVENTION

The present invention relates to the field of biopharmaceutical manufacturing. In particular, the invention relates to methods for reducing the formation of low molecular weight species of recombinantly-produced proteins during production cell culture and compositions produced by such methods.

### BACKGROUND OF THE INVENTION

Production of proteins by recombinant methods using engineered host cells can result in the generation of a wide array of variants of the protein product that must be monitored and/or controlled to ensure consistent product quality. Such product variants can arise from, for example, post-translational modifications (e.g. glycosylation, oxidation, deamidation, etc.) or alternative RNA splicing resulting in different messenger RNA (mRNA) transcripts encoding the protein. Protein product variants that have altered functional characteristics as compared to the desired protein product are categorized as product-related impurities. Because product-related impurities can affect the overall efficacy and/or safety of a protein drug product, product-related impurities often must be monitored and controlled to certain specified levels in the final protein drug product. One such type of product-related impurities is low molecular weight (LMW) species of the protein, which can include truncated forms of the protein expressed by the host cell, fragments of the protein resulting from proteolytic processing, or incomplete assembly of the polypeptide chains in the case of multi-chain proteins. Reduction of the amount and/or type of LMW species produced during the cell culture process is particularly useful as it can eliminate the need for additional downstream purification steps to remove the LMW species from the drug product. Thus, methods for reducing formation of LMW species of proteins during the cell culture production process are desirable.

### SUMMARY OF THE INVENTION

The present invention is based, in part, on the development of methods to eliminate or reduce the amount of LMW species of a protein expressed by a host cell during the cell culture production process. In some embodiments, the methods of the invention reduce LMW species of a protein by controlling the pH of the production cell culture. In other embodiments, the methods of the invention reduce the number and/or amount of LMW species of a protein by reducing or eliminating splice variant isoforms of the protein expressed by the host cell.

In certain embodiments, the present invention provides methods for producing a recombinant protein composition comprising a reduced amount of LMW species of the protein. In one such embodiment, the methods comprise culturing a mammalian cell expressing a nucleic acid encoding the protein in a cell culture medium for a period of time during which the protein is expressed and secreted by the mammalian cell, wherein the pH of the culture medium is maintained at about 6.90 or less; and recovering the expressed protein from the cell culture medium to obtain the recombinant protein composition, wherein the composition comprises less than 20% total LMW species of the protein. In some embodiments, the recombinant protein composition produced by the methods described herein may comprise less than 18% total LMW species of the recombinant protein, for example about 15% or less or about 10% or less, such as from about 2% to about 10%, about 1% to about 8%, or about 2% to about 6% total LMW species of the recombinant protein, optionally determined by a reduced capillary electrophoresis-sodium dodecyl sulfate method. In such embodiments, the recombinant protein composition can be harvested cell culture fluid. In certain embodiments, the LMW species comprises a splice variant isoform of the protein.

In some embodiments of the methods of the invention, the pH of the cell culture medium is maintained at a pH from about 6.70 to about 6.90, a pH from about 6.75 to about 6.85, or a pH of about 6.80. The pH of the cell culture medium is preferably maintained within these ranges for the duration of the production phase of the cell culture, which can be at least 3 days or at least 7 days. In some embodiments, the duration of the production phase of the cell culture is from about 7 days to about 14 days. In other embodiments, the duration of the production phase of the cell culture is from about 12 days to about 15 days. In certain embodiments, the recombinant protein compositions produced by the methods described herein comprise a reduced amount of total LMW species of the protein as compared to compositions of the same recombinant protein produced by transformed mammalian cells cultured in a culture medium maintained at a pH above 6.90, for example, at a pH of 7.00, 7.10, 7.20, 7.30, or 7.40.

In other embodiments, the present invention provides methods for reducing expression and secretion of alternative splice variant isoforms of a recombinant protein from a mammalian cell. In one embodiment, the methods comprise transfecting a mammalian cell with a nucleic acid comprising a first polynucleotide encoding a signal peptide and a second polynucleotide encoding the recombinant protein, wherein the first polynucleotide is in the same open reading frame as the second polynucleotide, wherein the first polynucleotide comprises a GGG codon encoding glycine for any glycine residue occurring within the six carboxy-terminal amino acids of the signal peptide; culturing the mammalian cell in a cell culture medium under conditions where the recombinant protein is expressed and secreted into the medium; and recovering the recombinant protein from the cell culture medium to obtain a recombinant protein composition. In such embodiments, the number and/or amount of alternative splice variant isoforms of a recombinant protein expressed by the mammalian cell may be reduced as compared to the number and/or amount of alternative splice variant isoforms expressed by a mammalian cell comprising a signal peptide encoding-polynucleotide comprising a glycine GGT codon for any glycine residue within the six C-terminal amino acids of the signal peptide. In some embodiments, the mammalian cell is cultured in a cell culture medium maintained at a pH of about 6.90 or less, for example, at a pH from about 6.70 to about 6.90, at a pH from about 6.75 to about 6.85, or at a pH of about 6.80.

In certain embodiments of the methods of the invention, the first polynucleotide encoding a signal peptide comprises a GGG codon encoding glycine for a glycine residue occurring as the fourth to last C-terminal amino acid of the signal peptide. In other embodiments, the first polynucleotide comprises a GGG codon encoding glycine for a glycine residue occurring as the sixth to last C-terminal amino acid of the signal peptide. In still other embodiments, the first polynucleotide comprises a GGG codon encoding glycine for each glycine residue occurring as the sixth to last and fourth to last C-terminal amino acid of the signal peptide. The nucleotide immediately preceding any glycine GGG codon in the first polynucleotide encoding a signal peptide may be a nucleotide other than adenine (A), such as cytosine (C), thymine (T) or guanine (G). In one particular embodiment, the nucleotide immediately preceding any glycine GGG codon in the first polynucleotide is cytosine (C). The first polynucleotide may encode any signal peptide suitable for promoting the secretion of the recombinant protein from the transfected mammalian cell. In some embodiments, the first polynucleotide encodes a signal peptide comprising the amino acid sequence of any one of SEQ ID NOs: 6-19. In one embodiment, the first polynucleotide encodes a signal peptide comprising the amino acid sequence of SEQ ID NO: 6. In a related embodiment, the first polynucleotide comprises the nucleotide sequence of SEQ ID NO: 20.

Various types of recombinant proteins can be produced by the methods of the invention including, but not limited to, cytokines, growth factors, hormones, muteins, fusion proteins, antibodies, antibody fragments, peptibodies, T-cell engaging molecules, and multi-specific antigen binding proteins. In some embodiments, the recombinant protein produced by methods of the invention is an antibody or binding fragment thereof. In other embodiments, the recombinant protein produced by the methods of the invention is a T-cell engaging molecule, e.g. a single chain T-cell engaging molecule, such as a single chain PSMA×CD3 T-cell engaging molecule. In one such embodiment, the recombinant protein is a single chain PSMA×CD3 T-cell engaging molecule comprising the amino acid sequence of SEQ ID NO: 1. In related embodiments, the nucleic acid encoding the single chain PSMA×CD3 T-cell engaging molecule comprises a nucleotide sequence of any one of SEQ ID NOs: 2-5. Thus, the present invention also includes isolated nucleic acids and expression vectors encoding the single chain PSMA×CD3 T-cell engaging molecule comprising a nucleotide sequence of any one of SEQ ID NOs: 2-5 as well as host cells, such as mammalian host cells (e.g. Chinese hamster ovary (CHO) cells), transformed with the isolated nucleic acids or expression vectors. In some embodiments, the present invention provides methods for producing a single chain PSMA×CD3 T-cell engaging molecule comprising culturing a mammalian host cell transformed with an isolated nucleic acid or expression vector comprising a nucleotide sequence of any one of SEQ ID NOs: 2-5 in a cell culture medium under conditions where the T-cell engaging molecule is expressed, and recovering the T-cell engaging molecule from the culture medium or host cell.

The present invention also includes recombinant protein compositions produced by the methods described herein. Such recombinant protein compositions have a reduced amount and/or variety of LMW species of the recombinant protein as compared to the amount and/or variety of LMW species of the recombinant protein produced by other cell culture methods. In certain embodiments, the present invention provides a composition comprising a single chain PSMA×CD3 T-cell engaging molecule and one or more LMW species thereof, wherein the composition comprises less than 20% total LMW species of the T-cell engaging molecule, and wherein the T-cell engaging molecule comprises the amino acid sequence of SEQ ID NO: 1. In certain embodiments, the compositions comprise less than 18% total LMW species of the T-cell engaging molecule, for example about 15% or less or about 10% or less, such as from about 2% to about 10%, about 1% to about 8%, or about 2% to about 6% total LMW species of the T-cell engaging molecule. In some embodiments, the LMW species comprises a splice variant isoform of the T-cell engaging molecule, such as a splice variant isoform comprising the sequence of SEQ ID NO: 23. The amount of the LMW species in the composition may be determined by a reduced capillary electrophoresis-sodium dodecyl sulfate method.

Pharmaceutical formulations comprising the single chain PSMA×CD3 T-cell engaging molecule compositions described herein are also included in the invention. In some embodiments, the pharmaceutical formulations comprise a single chain PSMA×CD3 T-cell engaging molecule composition described herein and one or more pharmaceutically acceptable excipients, such as buffers, sugars, and surfactants.

The present invention also includes methods for treating a PSMA-expressing cancer in a patient in need thereof using the single chain PSMA×CD3 T-cell engaging molecule compositions and pharmaceutical formulations comprising such compositions. In one embodiment, the methods comprise administering to the patient a pharmaceutical formulation comprising a single chain PSMA×CD3 T-cell engaging molecule composition described herein. The PSMA-expressing cancer can be prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, and melanoma. In certain embodiments, the PSMA-expressing cancer is prostate cancer, such as castration-resistant prostate cancer or metastatic castration-resistant prostate cancer.

The use of the single chain PSMA×CD3 T-cell engaging molecule compositions in any of the treatment methods or for preparation of medicaments for treating a PSMA-expressing cancer is specifically contemplated. For instance, the present invention encompasses a single chain PSMA×CD3 T-cell engaging molecule composition or pharmaceutical formulation described herein for use in a method for treating a PSMA-expressing cancer, such as prostate cancer, in a patient in need thereof. The present invention also includes the use of a single chain PSMA×CD3 T-cell engaging molecule composition or pharmaceutical formulation described herein in the preparation of a medicament for treating a PSMA-expressing cancer, such as prostate cancer, in a patient in need thereof.

### BRIEF DESCRIPTION OF THE DRAWINGS

FIG. 1 shows the relationship between the percentage of total LMW species of a PSMA×CD3 bispecific T-cell engager polypeptide in harvested cell culture fluid produced from two different CHO cell lines (process 1 and process 2) and the pH setpoint of the production bioreactor. LMW species of the polypeptide were measured by a reduced capillary electrophoresis-sodium dodecyl sulfate (rCE-SDS) method.

FIG. 2 depicts the mean viable cell density (10⁵ cells/mL) for a CHO cell line expressing a PSMA×CD3 bispecific T-cell engager polypeptide in production bioreactors operated at different setpoint pH values over days in culture. Error bars represent standard error of the mean.

FIG. 3 shows the mean percent cell viability for a CHO cell line expressing a PSMA×CD3 bispecific T-cell engager polypeptide in production bioreactors operated at different setpoint pH values over days in culture. Error bars represent standard error of the mean.

FIG. 4 depicts a representative chromatogram from a cation exchange chromatographic separation of a partially purified harvested cell culture fluid containing a PSMA×CD3 bispecific T-cell engager polypeptide. The separation was performed using a Capto-SP ImpRes® resin and an acetate pH 4.5 mobile phase with elution by a linear gradient of sodium chloride. Detection of proteins was by UV absorbance at 280 nm.

FIG. 5 shows the relationship between potency of PSMA×CD3 bispecific T-cell engager polypeptide drug substance samples in a cell-based activity assay (solid circles) or a binding assay (solid squares) and the percentage of LMW species present in the drug substance samples. Potency is plotted as % relative potency obtained by normalizing the activity of each of the drug substance samples to the activity of reference standard.

FIG. 6 is a Northern Blot analysis of RNA isolated from a cell line expressing a PSMA×CD3 bispecific T-cell engager polypeptide at different stages of cell line development. A smaller transcript variant was detected in all cell line stages in addition to the expected transcript at about 4.5 kb. Lane 1=untransfected host cell (negative control; HC (untrans.)); lane 2=pre-master cell bank (preMCB); lane 3=preMCB end of production (preMCB EOP); lane 4=mock working cell bank (mWCB); lane 5=mWCB end of production (mWCB EOP); lane 6=mock limit of in vitro cell age (mLIVCA); and lane 7=mLIVCA end of production (mLIVCA EOP).

FIG. 7 is a gel image showing the separation of reaction products from a cDNA RT-PCR analysis of RNA isolated from a cell line (MCB), clones, and pools of transfected CHO cells expressing a PSMA×CD3 bispecific T-cell engager polypeptide. All cells transfected with the nucleotide sequence set forth in SEQ ID NO: 2 expressed a shorter transcript variant (˜2.8 kb) as indicated by the white arrow in addition to the expected transcript at ˜3.4 kb. NTC=non-transfected control cell line.

FIG. 8 is a schematic depicting an alternative splicing event resulting in a transcript variant encoding a truncated form of a PSMA×CD3 bispecific T-cell engager polypeptide. A splice donor site in the signal peptide (SP) sequence and a splice acceptor site in the PSMA scFv sequence create a consensus splice site resulting in the deletion of 651 nucleotides from the 5′ end of the alternative transcript.

FIG. 9A is a Northern Blot analysis of RNA isolated from cell lines expressing a PSMA×CD3 bispecific T-cell engager polypeptide. The original cell line expressed a nucleotide sequence of SEQ ID NO: 2, whereas clones 059 and E13 expressed a modified nucleotide sequence of SEQ ID NO: 4. The first lane (HC (untrans.)) is RNA isolated from an untransfected host cell and represents a negative control. The smaller transcript variant detected in RNA isolated from the original cell line was not detectable in the two clones expressing the modified nucleotide sequence.

FIG. 9B is a gel image showing the separation of reaction products from a RT-PCR analysis of RNA isolated from cell lines expressing a PSMA×CD3 bispecific T-cell engager polypeptide. The original cell line expressed a nucleotide sequence of SEQ ID NO: 2, whereas clones 059 and E13 expressed a modified nucleotide sequence of SEQ ID NO: 4. The smaller transcript variant present in the original cell line is not detectable in the two clones expressing the modified nucleotide sequence.

FIG. 10 shows percent LMW species of a PSMA×CD3 bispecific T-cell engager polypeptide in harvested cell culture fluid (HCCF) obtained from different cell lines. The original cell line expressed a nucleotide sequence of SEQ ID NO: 2, whereas clones 059 and E13 expressed a modified nucleotide sequence of SEQ ID NO: 4. LMW species of the polypeptide were measured by a reduced capillary electrophoresis-sodium dodecyl sulfate (rCE-SDS) method.

### DETAILED DESCRIPTION

The present invention relates to methods for reducing the variety and/or amount of LMW species of a recombinant protein expressed by a host cell during the cell culture production process. LMW species of a protein product, which include truncated forms or fragments of the protein, typically have reduced functional activity compared to the desired protein product and often must be removed or controlled to within specific amounts to ensure the final protein drug product has the desired efficacy. Reduction of the variety and/or amount of LMW species produced during the cell culture phase of a recombinant protein manufacturing process can allow for a more streamlined downstream purification process by eliminating steps or unit operations designed to remove LMW species impurities. The methods of the invention can be used to produce recombinant protein compositions comprising less than 20% LMW species of the protein, for example without the need for further purification steps to remove the LMW species.

Any type of recombinant protein, including proteins containing single polypeptide chains or multiple polypeptide chains, can be produced according to the methods of the invention. The term “recombinant protein” refers to a heterologous protein produced by a host cell transfected with a nucleic acid encoding the protein when the host cell is cultivated in cell culture. Recombinant proteins can include, but are not limited to, cytokines, growth factors, hormones, muteins, fusion proteins, antibodies, antibody fragments, peptibodies, T-cell engaging molecules, and multi-specific antigen binding proteins. In some embodiments, the recombinant protein is a fusion protein. A “fusion protein” is a protein that contains at least one polypeptide fused or linked to a heterologous polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell to produce the fusion protein. The fusion protein may comprise a fragment from an immunoglobulin protein, such as an Fc region, fused or linked to a ligand polypeptide, a receptor polypeptide, a hormone, cytokine, growth factor, an enzyme, or other polypeptide that is not a component of an immunoglobulin.

In other embodiments, the recombinant protein to be produced according to the methods of the invention is an antibody or binding fragment thereof. As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus (N-terminus) to carboxyl terminus (C-terminus), a single immunoglobulin light chain variable region (VL) and a single immunoglobulin light chain constant domain (CL). The immunoglobulin light chain constant domain (CL) can be a human kappa (κ) or human lambda (2) constant domain. The term “heavy chain” or “immunoglobulin heavy chain” refers to a polypeptide comprising, from amino terminus (N-terminus) to carboxyl terminus (C-terminus), a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (u), delta (4), gamma (Y), alpha (a), and epsilon (¿), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

An “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VHH fragment), a Fab fragment, a Fab′ fragment, a F(ab′)₂ fragment, a Fv fragment, a Fd fragment, and a complementarity determining region (CDR) fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment. The “Fc fragment” or “Fc domain” of an immunoglobulin generally comprises two constant domains, a CH2 domain and a CH3 domain, and optionally comprises a CH4 domain.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VHH domain, in these animals. These antibodies form antigen-binding regions using only heavy chain variable regions, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold.

In embodiments in which the recombinant protein is an antibody or binding fragment thereof, the antibody can be a monoclonal antibody. The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from an animal (e.g. a transgenic animal expressing human immunoglobulin genes) after completion of an immunization schedule.

In some embodiments, the antibody (e.g. monoclonal antibody) or binding fragment thereof is a humanized antibody or binding fragment thereof. A “humanized antibody” refers to an antibody in which regions (e.g. framework regions) have been modified to comprise corresponding regions from a human immunoglobulin. Generally, a humanized antibody can be produced from a monoclonal antibody raised initially in a non-human animal, such as a rodent or rabbit. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent or rabbit variable region for the corresponding regions of a human antibody (see, e.g., U.S. Pat. Nos. 5,585,089 and 5,693,762; Jones et al., Nature, Vol. 321:522-525, 1986; Riechmann et al., Nature, Vol. 332:323-27, 1988; Verhoeyen et al., Science, Vol. 239:1534-1536, 1988). The CDRs of light and heavy chain variable regions of antibodies generated in another species can be grafted to consensus human framework regions (FRs) or FRs from specific human germline genes. To create consensus human FRs, FRs from several human heavy chain or light chain amino acid sequences may be aligned to identify a consensus amino acid sequence.

In other embodiments, the antibody (e.g. monoclonal antibody) or binding fragment thereof is a fully human antibody or binding fragment thereof. A “fully human antibody” is an antibody that comprises variable and constant regions derived from or indicative of human germ line immunoglobulin sequences. Fully human antibodies can be produced by immunizing transgenic animals (usually mice) that are capable of producing a repertoire of human antibodies in the absence of endogenous immunoglobulin production. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. One particular transgenic mouse line suitable for generation of fully human antibodies is the XenoMouse® transgenic mouse line described in U.S. Pat. Nos. 6,114,598; 6,162,963; 6,833,268; 7,049,426; 7,064,244; Green et al., 1994, Nature Genetics 7:13-21; Mendez et al., 1997, Nature Genetics 15:146-156; Green and Jakobovitis, 1998, J. Ex. Med, 188:483-495; Green, 1999, Journal of Immunological Methods 231:11-23; Kellerman and Green, 2002, Current Opinion in Biotechnology 13, 593-597. Additional methods relating to transgenic mice for making human antibodies are described in U.S. Pat. Nos. 5,545,807; 6,713,610; 6,673,986; 6,162,963; 5,939,598; 5,545,807; 6,300,129; 6,255,458; 5,877,397; 5,874,299 and 5,545,806; in PCT publications WO91/10741, WO90/04036, WO 94/02602, WO 96/30498, WO 98/24893 and in EP 546073B1 and EP 546073A1.

Antibodies, multi-specific antigen-binding proteins, and fusion proteins that may be produced according to the methods of the invention may bind to one or more target proteins including, but not limited to, CD2, CD3, CD4, CD8, CD11a, CD14, CD18, CD19, CD20, CD22, CD23, CD28, CD25, CD33, CD40, CD44, CD52, CD80 (B7.1), CD86 (B7.2), CD147, IL-1a, IL-1β, IL-4, IL-5, IL-8, IL-10, IL-13, IL-15, IL-2 receptor, IL-4 receptor, IL-6 receptor, IL-13 receptor, IL-18 receptor subunits, angiopoietin (e.g. angiopoietin-1, angiopoietin-2, or angiopoietin-4), platelet derived growth factor receptor beta (PDGF-β), vascular endothelial growth factor (VEGF), transforming growth factors (TGF), including, among others, TGF-α and TGF-β, including TGF-β1, TGF-β2, TGF-β3, TGF-β4, or TGF-β5, epidermal growth factor (EGF) receptor, VEGF receptor, HER2, FGF receptor, C5 complement, Beta-klotho, calcitonin gene-related peptide (CGRP), CGRP receptor, pituitary adenylate cyclase activating polypeptide (PACAP), pituitary adenylate cyclase activating polypeptide type 1 receptor (PACI receptor), IgE, tumor antigens, PD-1, PD-L1, HER-2, integrin alpha 4 beta 7, the integrin VLA-4, B2 integrins, TRAIL receptors 1,2,3, and 4, RANK, RANK ligand, sclerostin, Dickkopf-1 (DKK-1), TLA1, tumor necrosis factor alpha (TNF-«), epithelial cell adhesion molecule (EpCAM), intercellular adhesion molecule-3 (ICAM-3), leukointegrin adhesin, the platelet glycoprotein gp IIb/IIIa, cardiac myosin heavy chain, proprotein convertase subtilisin/Kexin Type 9 (PCSK9), thymic stromal lymphopoietin (TSLP), parathyroid hormone, rNAPc2, MHC I, carcinoembryonic antigen (CEA), alpha-fetoprotein (AFP), CTLA-4 (which is a cytotoxic T lymphocyte-associated antigen), Fc-γ-1 receptor, HLA-DR 10 beta, HLA-DR antigen, L-selectin, IPN-γ, respiratory syncytial virus, human immunodeficiency virus (HIV), hepatitis B virus (HBV), Streptococcus mutans, and Staphylococcus aureus.

In certain embodiments, the recombinant protein to be produced according to the methods of the invention is a T-cell engaging molecule. The term “T-cell engaging molecule” refers to a molecule that comprises at least one domain in which the structure is derived from or comprises the minimum structural features of an antibody, e.g., of a full-length immunoglobulin molecule, that allow for specific binding to an antigen on the surface of a T cell, such as cluster of differentiation 3 (CD3). Thus, a T-cell engaging molecule generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e. CDRL1, CDRL2 and CDRL3 of a VL region) and/or three heavy chain CDRs (i.e. CDRH1, CDRH2 and CDRH3 of a VH region), and preferably all six CDRs from both the light and heavy chain variable regions. The T-cell engaging molecules may comprise domains or regions (e.g. CDRs or variable regions) from monoclonal, chimeric, humanized and human antibodies. The T-cell engaging molecules produced according to the methods of the invention may comprise one or more polypeptide chains. In some embodiments, the T-cell engaging molecules are single-chain polypeptides. In other embodiments, the T-cell engaging molecules comprise two or more polypeptide chains—e.g. are polypeptide dimers or multimers. In certain embodiments, the T-cell engaging molecules comprise four polypeptide chains, and may, e.g. have the format of an antibody or an immunoglobulin protein.

The T-cell engaging molecules produced according to the methods of the invention may be at least bispecific T-cell engaging molecules. The term “bispecific T-cell engaging molecule” refers to a molecule capable of specifically binding to two different antigens. In the context of the present invention, such bispecific T-cell engaging molecules specifically bind to a cancer cell antigen (e.g. human cancer cell antigen) on the cell surface of target cells and CD3 (e.g. human CD3) on the cell surface of T cells. The bispecific T-cell engaging molecules produced according to the methods of the invention may specifically bind to CD3 (e.g. human CD3) on the surface of T cells and a target cancer cell antigen selected from 5T4, AFP, BCMA, beta-catenin, BRCA1, CD19, CD20, CD22, CD33, CD70, CD123, CDH19, CDK4, CEA, CLDN18.2, DLL3, DLL4, EGFR, EGFRvIII, EpCAM, EphA2, FLT3, FOLR1, gpA33, GPRC5D, HER2, IGFR, MAGE-1, MAGE-2, MAGE-3, MAGE-4, MAGE-6, MAGE-12, MSLN, MUC1, MUC2, MUC3, MUC4, MUC5, MUC16, MUC17, PSCA, PSMA, RAGE proteins, STEAP1, STEAP2, TRP1, and TRP2. In some embodiments, the bispecific T-cell engaging molecule is a single-chain polypeptide comprising a first scFv that specifically binds to a cancer cell antigen, such as any of the antigens described above, and a second scFv that specifically binds to CD3 (e.g. CD3 epsilon). In other embodiments, the bispecific T-cell engaging molecule is a single-chain polypeptide comprising a first scFv that specifically binds to a cancer cell antigen, such as any of the antigens described above, a second scFv that specifically binds to CD3 (e.g. CD3 epsilon), and a single-chain Fc domain (scFc domain).

An antibody or binding fragment thereof, multi-specific antigen-binding protein, fusion protein, or T-cell engaging molecule or binding domain thereof “specifically binds” to a target antigen when it has a significantly higher binding affinity for, and consequently is capable of distinguishing, that antigen compared to its affinity for other unrelated proteins, under similar binding assay conditions. Antibodies or binding fragments thereof, multi-specific antigen-binding proteins, fusion proteins, or T-cell engaging molecules or binding domains thereof that specifically bind an antigen may bind to that antigen with an equilibrium dissociation constant (K_{D})≤1×10⁻⁶ M. Antibodies or binding fragments thereof, multi-specific antigen-binding proteins, fusion proteins, or T-cell engaging molecules or binding domains thereof specifically bind antigen with “high affinity” when the K_{D} is ≤1×10⁻⁸ M. Binding affinity can be determined using a variety of techniques, including affinity ELISA, surface plasmon resonance (e.g., with a BIAcore® instrument), a Kinetic Exclusion Assay (KinExA) as described in Rathanaswami et al. Analytical Biochemistry, Vol. 373:52-60, 2008, and bio-layer interferometry, such as that described in Kumaraswamy et al., Methods Mol. Biol., Vol. 1278:165-82, 2015 and employed in Octet® systems (Pall ForteBio).

In certain embodiments, the recombinant protein to be produced according to the methods of the invention is a bispecific T-cell engaging molecule comprising a first binding domain that specifically binds to prostate specific membrane antigen (PSMA) and a second binding domain that specifically binds to CD3 epsilon. Examples of such PSMA×CD3 bispecific T-cell engaging molecules that can be produced according to the methods of the invention are described in, for example, WO 2010/037836, WO 2017/023761, WO 2017/121905, WO 2017/134158, WO 2018/098356, WO 2019/224718, and WO 2020/206330, all of which are hereby incorporated by reference in their entireties. In some embodiments, the PSMA×CD3 bispecific T-cell engaging molecule produced according to the methods of the invention is a single chain T-cell engaging molecule. As used herein, a “single chain T-cell engaging molecule” or “single chain T-cell engaging polypeptide” refers to a molecule consisting of only one polypeptide chain, i.e. all of the domains in the bispecific T-cell engaging molecule are linked together, optionally via peptide linkers, to form a single polypeptide chain. One example of such a single chain PSMA×CD3 bispecific T-cell engaging molecule in the context of the present invention is a single chain polypeptide comprising, in an amino to carboxyl order, an anti-PSMA scFv domain, a first peptide linker, an anti-CD3 scFv domain, a second peptide linker, and an scFc domain, such as the molecules described in WO 2017/134158. In one embodiment, the recombinant protein to be produced according to the methods of the invention is a single chain bispecific T-cell engaging polypeptide comprising the amino acid sequence of SEQ ID NO: 1. Nucleic acids encoding this single chain PSMA×CD3 bispecific T-cell engaging polypeptide are described in further detail herein and include the nucleotide sequences set forth in SEQ ID NOs: 2-5.

The methods of the invention reduce the variety and/or amount of LMW species of a recombinant protein expressed or produced by a host cell during the cell culture production process. LMW species of a recombinant protein refer to fragments, truncated forms, or other incomplete variants of the recombinant protein that have a molecular weight less than the molecular weight of the intact, fully assembled form of the recombinant protein. LMW species can include, but are not limited to, proteolytic fragments, truncated forms resulting from cellular expression of mRNA splice variants, and single component polypeptides in the case of multi-polypeptide chain proteins (e.g. light chain or heavy chain only species when the recombinant protein is an antibody).

In certain embodiments, the present invention provides a method for producing a recombinant protein composition comprising a reduced amount of LMW species of the protein, the method comprising culturing a mammalian cell expressing a nucleic acid encoding the protein in a cell culture medium for a period of time during which the protein is expressed and secreted by the mammalian cell, wherein the pH of the culture medium is maintained at about 6.90 or less; and recovering the expressed protein from the cell culture medium to obtain the recombinant protein composition, wherein the composition comprises less than 20% total LMW species of the protein.

To generate mammalian cell lines engineered to express the recombinant protein of interest, one or more nucleic acids encoding the recombinant protein (or components thereof in the case of multi-chain proteins) is initially inserted into one or more expression vectors. The term “expression vector” or “expression construct” as used herein refers to a recombinant DNA molecule containing a desired coding sequence and appropriate nucleic acid control sequences necessary for the expression of the operably linked coding sequence in a particular host cell, e.g. a mammalian host cell. Vectors can include viral vectors, non-episomal mammalian vectors, plasmids and other non-viral vectors. An expression vector can include sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein. Nucleic acid control sequences useful in expression vectors for expression in mammalian cells include promoters, enhancers, and termination and polyadenylation signals. A secretory signal peptide sequence can also, optionally, be encoded by the expression vector, operably linked to the coding sequence of interest, so that the expressed protein can be secreted by the recombinant host cell, for more facile isolation of the recombinant protein from the cell, if desired. Vectors may also include one or more selectable marker genes to facilitate selection of host cells into which the vectors have been introduced. In some embodiments, vectors are used that employ protein-fragment complementation assays using protein reporters, such as dihydrofolate reductase (see, for example, U.S. Pat. No. 6,270,964). Suitable mammalian expression vectors are known in the art and are also commercially available.

Typically, vectors used in any of the host cells will contain sequences for plasmid maintenance and for cloning and expression of exogenous nucleotide sequences. Such sequences will typically include one or more of the following nucleotide sequences: a promoter, one or more enhancer sequences, an origin of replication, transcriptional and translational control sequences, a transcriptional termination sequence, a complete intron sequence containing a donor and acceptor splice site, a native or heterologous signal peptide sequence (leader sequence or signal peptide) for polypeptide secretion, a ribosome binding site, a polyadenylation sequence, a polylinker region for inserting the polynucleotide encoding the polypeptide to be expressed, and a selectable marker element. Vectors may be constructed from a starting vector such as a commercially available vector, and additional elements may be individually obtained and ligated into the vector.

Vector components may be homologous (i.e., from the same species and/or strain as the host cell), heterologous (i.e., from a species other than the host cell species or strain), hybrid (i.e., a combination of flanking sequences from more than one source), synthetic or native. The sequences of components useful in the vectors may be obtained by methods well known in the art, such as those previously identified by mapping and/or by restriction endonuclease digestion. In addition, they can be obtained by polymerase chain reaction (PCR) and/or by screening a genomic library with suitable probes.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

An origin of replication aids in the amplification of the vector in a host cell. They may be included as part of commercially available prokaryotic vectors and may also be chemically synthesized based on a known sequence and ligated into the vector. Various viral origins (e.g., SV40, polyoma, adenovirus, vesicular stomatitus virus (VSV), or papillomaviruses such as HPV or BPV) are useful for cloning vectors in mammalian cells.

Expression and cloning vectors used in the methods of the invention will typically contain a promoter that is recognized by the host organism and operably linked to the polynucleotide encoding the polypeptide. Promoters are non-transcribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from polynucleotides under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the polynucleotide encoding a recombinant protein by removing the promoter from the source nucleic acid by restriction enzyme digestion and inserting the desired promoter sequence into the vector. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

An enhancer sequence may be inserted into the vector to increase transcription of a polynucleotide encoding a recombinant protein by higher eukaryotes. Enhancers are cis-acting elements of nucleic acid, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter.

A sequence encoding an appropriate native or heterologous signal peptide sequence (leader sequence or signal peptide) can be incorporated into an expression vector, to promote extracellular secretion of the recombinant protein. The choice of signal peptide or leader depends on the type of host cells in which the recombinant protein is to be produced, and a heterologous signal sequence can replace the native signal sequence. Examples of signal peptides are described in more detail herein. Other signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367566; the type I interleukin-1 receptor signal peptide described in U.S. Pat. No. 4,968,607; and the type II interleukin-1 receptor signal peptide described in EP Patent No. 0460846.

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis known to those of skill in the art.

Exemplary transcriptional and translational control sequences for mammalian host cell expression vectors can be excised from viral genomes. Commonly used promoter and enhancer sequences are derived from polyoma virus, adenovirus 2, simian virus 40 (SV40), and human cytomegalovirus (CMV). For example, the human CMV promoter/enhancer of immediate early gene 1 may be used. See e.g. Patterson et al. (1994), Applied Microbiol. Biotechnol. 40:691-98. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al. (1978), Nature 273:113; Kaufman (1990), Meth. in Enzymol. 185:487-511). Smaller or larger SV40 fragments can also be used, provided the approximately 250 bp sequence extending from the Hind III site toward the Bgl I site located in the SV40 viral origin of replication site is included.

A selectable marker gene encoding a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include glutamine synthase (GS)/methionine sulfoximine (MSX) system, dihydrofolate reductase (DHFR), and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes a protein of interest. As a result, increased quantities of a polypeptide of interest are synthesized from the amplified DNA.

After the expression vector(s) has been constructed and the one or more nucleic acid molecules encoding the recombinant protein (or components thereof in the case of multi-chain proteins) has been inserted into the proper site(s) of the vector or vectors, the completed vector(s) may be inserted into a suitable host cell for amplification and/or polypeptide expression. The transformation of an expression vector into a selected host cell may be accomplished by well-known methods including transfection, transduction, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth in manuals and other technical publications, for example, in Sambrook et al. Molecular Cloning; A Laboratory Manual, Third Edition, Cold Spring Harbor Laboratory Press, Cold Spring Harbor, N.Y., (2001), and Ausubel et al. (eds.) Current Protocols in Molecular Biology, Greene Publishing Associates, (1989).

As used herein, the term “transformation” refers to a change in a cell's genetic characteristics, and a cell is considered to have been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell or can be maintained transiently as an episomal element without being replicated, or can replicate independently as a plasmid. A cell is considered to have been “stably transformed” when the transforming DNA is replicated with the division of the cell.

As used herein, the term “transfection” refers to the uptake of foreign or exogenous DNA by a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. As used herein, the term “transduction” refers to the process whereby foreign DNA is introduced into a cell via viral vector. See Jones et al., (1998). Genetics: principles and analysis. Boston: Jones & Bartlett Publ.

The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises a nucleic acid encoding a recombinant protein, preferably operably linked to at least one expression control sequence (e.g. promoter or enhancer), is a “recombinant host cell.” A host cell, when cultured under appropriate conditions, synthesizes the recombinant protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. In certain embodiments of the methods of the invention, the host cell is a mammalian host cell.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa, Schwanniomyces, such as Schwanniomyces occidentalis; and filamentous fungi, such as, e.g., Neurospora, Penicillium, Tolypocladium, and Aspergillus hosts such as A. nidulans and A. niger.

Vertebrate host cells are also suitable hosts for expressing recombinant proteins. Mammalian cell lines suitable as hosts for recombinant protein expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. CHO cells are preferred mammalian host cells in some embodiments of the methods of the invention for expressing recombinant proteins.

In certain embodiments, the methods of the invention comprise culturing the transformed host cell (e.g. transformed mammalian host cell) in a cell culture medium under conditions and for a period of time during which the recombinant protein is expressed and secreted by the mammalian host cell. The term “culture” or “culturing” refers to the growth and propagation of cells outside of a multicellular organism or tissue. Host cells may be cultured in suspension or in an adherent form, attached to a solid substrate. Cell cultures can be established in fluidized bed bioreactors, hollow fiber bioreactors, roller bottles, shake flasks, or stirred tank bioreactors, with or without microcarriers. In some embodiments, the transformed mammalian cells, such as transformed CHO cells, may be cultured in production bioreactors at a small scale, for example, at a volume of 5 liters or less, 3 liters or less, or 1 liter or less. In other embodiments, the transformed mammalian cells (e.g. transformed CHO cells) are cultured in production bioreactors with a capacity of at least 500 liters, at least 1,000 liters, at least 2,000 liters, at least 5,000 liters, at least 10,000 liters, or at least 15,000 liters. Such production cell cultures may be maintained for several weeks and even months, during which the cells produce the desired recombinant protein.

Suitable culture conditions, including temperature, dissolved oxygen content, agitation rate, and the like, for mammalian cells are known in the art and may vary by the phase or stage of the cell culture. The “growth phase” of a cell culture refers to the period of exponential cell growth (i.e. the log phase) where cells are generally rapidly dividing. During the growth phase, cells are cultured in a cell culture medium containing the necessary nutrients and additives under conditions (generally at about a temperature of 25°-40° C., in a humidified, controlled atmosphere) such that optimal growth is achieved for the particular cell line. Cells are typically maintained in the growth phase for a period of between one and eight days, e.g., between three to seven days, e.g., seven days. The length of the growth phase for a particular cell line can be determined by a person of ordinary skill in the art and will generally be the period of time sufficient to allow the particular cells to reproduce to a viable cell density within a range of about 20%-80% of the maximal possible viable cell density if the culture was maintained under the growth conditions. A “production phase” of a cell culture refers to the period of time during which logarithmic cell growth has ended and recombinant protein production is predominant. During the production phase, the medium is generally supplemented to support continued recombinant protein production.

In certain embodiments of the methods of the invention, the culture conditions may be adjusted to facilitate the transition from the growth phase of the cell culture to the production phase. For instance, a growth phase of the cell culture may occur at a higher temperature than a production phase of the cell culture. In some embodiments, a growth phase may occur at a first temperature from about 35° C. to about 38° C., and a production phase may occur at a second temperature from about 29° ° C. to about 37° C., optionally from about 30° C. to about 36° C. or from about 30° ° C. to about 34ºC. In one embodiment, a shift in temperature from about 35° C. to about 37° C. to a temperature of about 31° C. to about 33° C. may be employed to facilitate the transition from the growth phase of the culture to the production phase. Chemical inducers of protein production, such as, for example, caffeine, butyrate, and hexamethylene bisacetamide (HMBA), may be added at the same time as, before, and/or after a temperature shift, or in place of a temperature shift. If inducers are added after a temperature shift, they can be added from one hour to five days after the temperature shift, optionally from one to two days after the temperature shift.

Cell culture media, as the term is used herein, refers to a solution containing nutrients sufficient to sustain growth and survival of a host cell during in vitro cell culture. Typically, cell culture media contains a buffer, salts, energy source, amino acids, vitamins and trace essential elements. Any media capable of supporting growth of the appropriate host cell in culture can be used. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture media may also be enriched by the addition of nutrients or other supplements, which may be used at greater than usual, recommended concentrations. In certain embodiments, the culture medium used in the methods of the invention is a chemically defined medium, which refers to a cell culture medium in which all of the components have known chemical structures and concentrations. Chemically defined media are typically serum-free and do not contain hydrolysates or animal-derived components.

Various media formulations can be used during the life of the culture, for example, to facilitate the transition from one stage (e.g., the growth stage or phase) to another (e.g., the production stage or phase) and/or to optimize conditions during cell culture (e.g. concentrated media provided during a perfusion culture). A growth medium formulation can be used to promote cell growth and minimize protein expression. A production medium formulation can be used to promote production of the recombinant protein of interest and maintenance of the cells, with minimal new cell growth). A feed media, typically a media containing more concentrated components such as nutrients and amino acids, which are consumed during the course of the production phase of the cell culture may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

In the methods of the invention, the mammalian cell can be cultured in a batch, fed batch, or perfusion culture. A “batch culture” refers to a method of culturing cells in which all the components required to establish the cell culture, including the transformed host cells, culture medium, and nutrients, are provided to the culture vessel at the beginning of the culturing process and no supplementation of the culture occurs. A batch culture is typically stopped at some point and the cells and/or components in the medium are harvested and recovered recombinant protein optionally purified. A “fed-batch culture” refers to a method of culturing cells in which additional components or nutrients (e.g. feed medium) are provided to the culture at one or more discrete times subsequent to the beginning of the culture process. A fed-batch culture is typically stopped at some point and the cells and/or components in the medium are harvested and the recombinant protein optionally purified. A “perfusion culture” refers to a method of culturing cells in which additional components or nutrients (e.g. feed medium) are provided continuously or semi-continuously to the culture subsequent to the beginning of the culture process. A portion of the cells and/or components in the medium are typically removed on a continuous or semi-continuous basis in a perfusion culture. In certain embodiments of the methods of the invention, the transformed mammalian cell is cultured in a perfusion culture.

In some embodiments of the methods of the invention, the mammalian cell is cultured to a viable cell density of at least 100×10⁵ cells/mL, for example between about 100×10⁵ cells/mL and about 10×10⁷ cells/mL, between about 250×10⁵ cells/mL and about 900×10⁵ cells/mL, between about 300×10⁵ cells/mL and 800×10⁵ cells/mL, or between about 450×10⁵ cells/mL and 650×10⁵ cells/mL. Cell density may be measured using a hemacytometer, a Coulter counter, or an automated cell analyzer (e.g. Cedex automated cell counter). Viable cell density may be determined by staining a culture sample with Trypan blue, which is taken up only by dead cells. Viable cell density is then determined by counting the total number of cells, dividing the number of stained cells by the total number of cells, and taking the reciprocal.

As described in the Examples, it was unexpectedly found that maintenance of the cell culture medium within a certain pH range during the production phase of the cell culture resulted in reduced LMW species of the recombinant protein produced by the transformed mammalian cell. Accordingly, in some embodiments, the methods of the invention comprise culturing a mammalian cell expressing a nucleic acid encoding a recombinant protein in a cell culture medium, wherein the pH of the cell culture medium is maintained at about 6.90 or less. In certain embodiments, the pH of the cell culture medium during the production phase of the cell culture is maintained at a pH from about 6.70 to about 6.90, for example, from about 6.70 to about 6.80, from about 6.75 to about 6.85, from about 6.78 to about 6.82, from about 6.80 to about 6.90, or from about 6.85 to about 6.90. In one embodiment, the pH of the cell culture medium is maintained at about 6.70. In another embodiment, the pH of the cell culture medium is maintained at about 6.80. In still another embodiment, the pH of the cell culture medium is maintained at about 6.90. In certain embodiments, the recombinant protein compositions produced by the methods of the invention comprise a reduced amount of total LMW species of the protein as compared to compositions of the same recombinant protein produced by transformed mammalian cells cultured in a culture medium maintained at a pH above 6.90, for example, at a pH of 7.00, 7.10, 7.20, 7.30, or 7.40.

In the methods of the invention, the mammalian cell is cultured for a defined period of time during which the recombinant protein is expressed and secreted by the mammalian cell. This period of time (i.e. the duration of the production phase of the cell culture) is at least 3 days, at least 7 days, at least 10 days, or at least 15 days. In certain embodiments, the duration of the production phase of the cell culture is about 7 days to about 28 days, about 10 days to about 30 days, about 7 days to about 14 days, about 10 days to about 18 days, about 3 days to about 15 days, about 5 days to about 8 days, about 12 days to about 15 days, about 12 days to about 18 days, or about 15 days to about 21 days. In some embodiments, the duration of the production phase of the cell culture is 7 days, 8 days, 9 days, 12 days, 15 days, 18 days, or 21 days. Preferably, the pH of the cell culture medium is maintained within the ranges described above for the entire duration of the production phase of the cell culture.

The methods of the invention further comprise recovering the expressed recombinant protein from the host cells (e.g. mammalian cells) or cell culture medium to obtain a recombinant protein composition. If the recombinant protein is produced intracellularly (i.e. is not secreted by the mammalian host cell), as a first step, the host cells are lysed (e.g., by mechanical shear, osmotic shock, or enzymatic methods) and the particulate debris (e.g., host cells and lysed fragments), is removed, for example, by centrifugation, flocculation, acoustic wave separation, or filtration, including, for example, by microfiltration, ultrafiltration, tangential flow filtration, alternative tangential flow filtration, and depth filtration. In certain preferred embodiments, the recombinant protein is secreted into the culture medium by the host cell (e.g. mammalian host cell). In such embodiments, the recombinant protein can be separated from host cells through centrifugation or microfiltration, and optionally, subsequently concentrated through ultrafiltration. In some embodiments, the expressed recombinant protein is recovered from the cell culture medium by microfiltration. In these and other embodiments, the expressed recombinant protein is recovered from the cell culture medium by alternating tangential flow filtration.

In some embodiments of the methods of the invention, the recombinant protein recovered from the host cells or cell culture medium may be further purified or partially purified to remove cell culture media components, host cell proteins or nucleic acids, or other process or product-related impurities by one or more unit operations. The term “unit operation” refers to a functional step that is performed as part of a process of purifying a recombinant protein of interest. For example, a unit operation can include steps such as, but not limited to, capturing, purifying, polishing, viral inactivating, virus filtering, concentrating and/or formulating the recombinant protein of interest. Unit operations can be designed to achieve a single objective or multiple objectives, such as capture and virus inactivating steps. Unit operations can also include holding or storing steps between processing steps. One of ordinary skill in the art can select the appropriate unit operation(s) for further purification of a recombinant protein based on the characteristics of the recombinant protein to be purified, the characteristics of host cell from which the recombinant protein is expressed, and the composition of the culture medium in which the host cells were grown.

A capture unit operation may include capture chromatography that makes use of resins and/or membranes containing agents that will bind to the recombinant protein of interest, for example affinity chromatography, size exclusion chromatography, ion exchange chromatography, hydrophobic interaction chromatography (HIC), immobilized metal affinity chromatography (IMAC), and the like. Such chromatographic materials are known in the art and are commercially available. For instance, if the recombinant protein is an antibody or contains components derived from an antibody (e.g. Fc domain), affinity chromatography using ligands such as Protein A, Protein G, Protein A/G, or Protein L may be employed as a capture chromatography unit operation to further purify the recombinant protein. In other embodiments, the recombinant protein of interest may comprise a polyhistidine tag at its amino or carboxyl terminus and subsequently purified using IMAC. Recombinant proteins can be engineered to include other purification tags, such as a FLAG® tag or c-myc epitope and subsequently purified by affinity chromatography using a specific antibody directed to such tag or epitope.

Unit operations to inactivate, reduce and/or eliminate viral contaminants may include filtration processes and/or adjusting solution conditions. One method for achieving viral inactivation is incubation at low pH (e.g., pH<4). A low pH viral inactivation operation can be followed with a neutralization unit operation that readjusts the virally inactivated solution to a pH more compatible with the requirements of the subsequent unit operations. A low pH viral inactivation operation may also be followed by filtration, such as depth filtration, to remove any resulting turbidity or precipitation. Adjusting the temperature or chemical composition (e.g. use of detergents) can also be used to achieve viral inactivation. Viral filtration can be performed using micro- or nano-filters, such as those available from Asahi Kasei (Plavona®) and EDM Millipore (VPro®).

A polishing unit operation may make use of various chromatographic methods for the purification of the protein of interest and clearance of contaminants and impurities. The polish chromatography unit operation makes use of resins and/or membranes containing agents that can be used in either a “flow-through mode,” in which the protein of interest is contained in the eluent and the contaminants and impurities are bound to the chromatographic medium, or “bind and elute mode,” in which the protein of interest is bound to the chromatographic medium and eluted after the contaminants and impurities have flowed through or been washed off the chromatographic medium. Examples of such polish chromatography methods include, but are not limited to, ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography, and size-exclusion chromatography (e.g. gel filtration).

Product concentration and buffer exchange of the recombinant protein of interest into a desired formulation buffer for bulk storage of the drug substance or drug product can be accomplished by ultrafiltration and diafiltration.

The recombinant protein compositions produced by the methods of the invention preferably comprise less than 20% total LMW species of the recombinant protein. As described in detail herein, the methods of the invention reduce the variety and/or amount of LMW species of a recombinant protein produced by a host cell during the cell culture process and thus obviate the need for downstream unit operations designed to specifically remove such LMW species. In certain embodiments, the recombinant protein composition is a harvested cell culture fluid. The term “harvested cell culture fluid” refers to a solution which has been processed by one or more operations to separate cells, cell debris, or other large particulates from the recombinant protein. Such operations, as described above, include, but are not limited to, flocculation, centrifugation, acoustic wave separation, and various forms of filtration (e.g. depth filtration, microfiltration, ultrafiltration, tangential flow filtration, and alternating tangential flow filtration). Harvested cell culture fluid includes cell culture lysates as well as cell culture supernatants. The harvested cell culture fluid may be further clarified to remove fine particulate matter and soluble aggregates by filtration with a membrane having a pore size between about 0.1 μm and about 0.5 μm, or more preferably a membrane having a pore size of about 0.22 μm. Thus, in some embodiments, the recombinant protein composition is a clarified harvested cell culture fluid.

In some embodiments, the recombinant protein compositions produced by the methods of the invention comprise less than 18% total LMW species of the recombinant protein, for example about 15% or less, about 12% or less, about 10% or less, about 8% or less, or about 6% or less total LMW species of the recombinant protein. In certain embodiments, the recombinant protein compositions produced by the methods of the invention comprise about 1% to about 18% total LMW species of the recombinant protein, such as about 5% to about 15%, about 2% to about 10%, about 1% to about 8%, or about 2% to about 6% total LMW species of the recombinant protein. In certain embodiments, the LMW species comprises a splice variant isoform of the protein. As used herein, a “splice variant isoform” refers to a variant of a protein translated from an alternatively spliced mRNA generated from the recombinant gene encoding the protein. A splice variant isoform will typically have a different amino acid sequence than that of the intended recombinant protein and is often a truncated form of the recombinant protein.

LMW species of a recombinant protein can be detected and quantitated using standard reduced capillary electrophoresis-sodium dodecyl sulfate methods (rCE-SDS). An exemplary rCE-SDS method suitable for measuring LMW species of a recombinant protein is described in Example 1. Other methods of detecting and quantitating LMW species of a recombinant protein are known to those of ordinary skill in the art and can include size exclusion chromatography (e.g. size exclusion-high performance liquid chromatography (SE-HPLC)), sedimentation velocity ultracentrifugation, and SE-HPLC with static light scattering detection to determine molar mass.

The present invention also provides a method for reducing expression and secretion of alternative splice variant isoforms of a recombinant protein from a mammalian cell. LMW species of a recombinant protein can arise from expression of unwanted mRNA splice variants by the transformed host cell during the cell culture process. As described in Example 3, it was discovered that use of the GGT codon to encode for a glycine residue at the carboxy terminal (i.e. C-terminal) end of a secretory signal peptide created a strong splice donor site resulting in an alternative splicing event leading to the generation of a truncated form of the recombinant protein. Replacement of the GGT codon with the GGG codon to encode the glycine residue at the C-terminal end of the signal peptide eliminated the alternative splice variant and reduced the amount of LMW species produced by the transformed host cell. Accordingly, in certain embodiments, the present invention includes a method for reducing expression and secretion of alternative splice variant isoforms of a recombinant protein from a mammalian cell, the method comprising: transfecting a mammalian cell with a nucleic acid comprising a first polynucleotide encoding a signal peptide and a second polynucleotide encoding the recombinant protein, wherein the first polynucleotide is in the same open reading frame as the second polynucleotide, wherein the first polynucleotide comprises a GGG codon encoding glycine for any glycine residue occurring within the six carboxy-terminal amino acids of the signal peptide; culturing the mammalian cell in a cell culture medium under conditions where the recombinant protein is expressed and secreted into the medium; and recovering the recombinant protein from the cell culture medium to obtain a recombinant protein composition. This method can be combined with the methods described above in which the transformed mammalian cell is cultured in a cell culture medium maintained at a pH of about 6.90 or less to further reduce the LMW species of the recombinant protein expressed by the transformed mammalian cell.

As described above, polynucleotides encoding secretory signal peptides are often incorporated into expression vectors for producing recombinant proteins to promote secretion of the recombinant protein by the host cell, thereby allowing recovery of the recombinant protein directly from the culture medium. A glycine codon GGT occurring within the six C-terminal amino acids of a signal peptide is poised to serve as a splice donor site that can be matched with a splice acceptor site that happens to be present in the nucleotide sequence encoding the recombinant protein. Due to the position of this potential splice donor site within the C-terminal end of the signal sequence, any alternative splicing event that may occur is likely to result in a truncated form of the recombinant protein. Therefore, use of a polynucleotide encoding a signal peptide comprising a glycine GGG codon for any glycine residue occurring within the six C-terminal amino acids of the signal peptide reduces the likelihood of unwanted splicing events by eliminating the strong splice donor site.

Thus, in certain embodiments, the methods comprise transfecting a mammalian cell with a nucleic acid comprising a first polynucleotide encoding a signal peptide and a second polypeptide encoding a recombinant protein, wherein the first polynucleotide comprises a GGG codon encoding glycine for any glycine residue occurring within the six C-terminal amino acids of the signal peptide. “Carboxy-terminal,” “carboxyl-terminal,” or “C-terminal” refers to the amino acids positioned at the end of a polypeptide chain terminating in a free carboxyl group (—COOH). Therefore, an amino acid within the six C-terminal amino acids of a polypeptide chain is an amino acid that is the sixth to last, fifth to last, fourth to last, third to last, second to last, or the last amino acid in the polypeptide chain. In certain embodiments, the first polynucleotide comprises a GGG codon encoding glycine for a glycine residue occurring as the fourth to last C-terminal amino acid of the signal peptide. In some embodiments, the first polynucleotide comprises a GGG codon encoding glycine for a glycine residue occurring as the last C-terminal amino acid of the signal peptide. In other embodiments, the first polynucleotide comprises a GGG codon encoding glycine for a glycine residue occurring as the sixth to last C-terminal amino acid of the signal peptide. In still other embodiments, the first polynucleotide comprises a GGG codon encoding glycine for each glycine residue occurring as the sixth to last and fourth to last C-terminal amino acid of the signal peptide. In any of the above-described embodiments, the nucleotide immediately preceding any glycine GGG codon may be a nucleotide other than adenine (A) (e.g. cytosine (C), thymine (T) or guanine (G)). In one embodiment, the nucleotide immediately preceding any glycine GGG codon is cytosine (C).

Exemplary signal peptides that can be encoded by the first polynucleotide include, but are not limited to MDMRVPAQLLGLLLLWLRGARC (SEQ ID NO: 6), MAWALLLLTLLTQGTGSWA (SEQ ID NO: 7), MTCSPLLLTLLIHCTGSWA (SEQ ID NO: 8), MEWTWRVLFLVAAATGAHS (SEQ ID NO: 9), MEWSWVFLFFLSVTTGVHS (SEQ ID NO: 10), MDIRAPTQLLGLLLLWLPGAKC (SEQ ID NO: 11), MDIRAPTQLLGLLLLWLPGARC (SEQ ID NO: 12), MDMRAPTQLLGLLLLWLPGARC (SEQ ID NO: 13), MDTRAPTQLLGLLLLWLPGATF (SEQ ID NO: 14), MDTRAPTQLLGLLLLWLPGARC (SEQ ID NO: 15), METGLRWLLLVAVLKGVQC (SEQ ID NO: 16), METGLRWLLLVAVLKGVQCQE (SEQ ID NO: 17), MEAPAQLLFLLLLWLPDTTG (SEQ ID NO: 18), and METPAQLLFLLLLWLPDTTG (SEQ ID NO: 19). In some embodiments, the first polynucleotide encodes a signal peptide comprising the amino acid sequence of SEQ ID NO: 6. In other embodiments, the first polynucleotide encodes a signal peptide comprising the amino acid sequence of SEQ ID NO: 7. In still other embodiments, the first polynucleotide encodes a signal peptide comprising the amino acid sequence of SEQ ID NO: 8. The first polynucleotide can comprise a nucleotide sequence encoding the amino acid sequence of any of the above-described signal peptides provided that the codon encoding glycine for any glycine occurring within the six C-terminal amino acids is GGG. In one embodiment, the first polynucleotide comprises the nucleotide sequence of SEQ ID NO: 20. In another embodiment, the first polynucleotide comprises the nucleotide sequence of SEQ ID NO: 21. In yet another embodiment, the first polynucleotide comprises the nucleotide sequence of SEQ ID NO: 22.

The second polynucleotide can encode any desired recombinant protein, such as the recombinant proteins described herein. In some embodiments, the recombinant protein is an antibody or binding fragment thereof. In other embodiments, the recombinant protein is a light chain or heavy chain of an antibody. In yet other embodiments, the recombinant protein is a fusion protein. In certain other embodiments, the recombinant protein is a T-cell engaging molecule, for example, a single chain T-cell engaging molecule. In related embodiments, the recombinant protein is a single chain PSMA×CD3 bispecific T-cell engaging molecule. In one such embodiment, the recombinant protein comprises the amino acid sequence of SEQ ID NO: 1. Thus, in certain embodiments, the second polynucleotide encodes a recombinant protein comprising the amino acid sequence of SEQ ID NO: 1. In one such embodiment, the second polynucleotide comprises the nucleotide sequence of SEQ ID NO: 5. In these and other embodiments, the nucleic acid, which comprises the first polynucleotide encoding a signal peptide and a second polynucleotide encoding a recombinant protein, comprises the nucleotide sequence of SEQ ID NO: 4.

Preferably, the nucleic acid comprises the first polynucleotide encoding the signal peptide in the same open reading frame as the second polynucleotide encoding the recombinant protein. The term “open reading frame” refers to a contiguous stretch of codons beginning at a start codon (e.g. ATG in DNA or AUG in RNA) and ending at a stop codon (e.g. TAA, TGA, and TAG in DNA or UAA, UGA, and UAG in RNA) that is translated into a polypeptide. Thus, when the first polynucleotide and second polynucleotide are positioned in the same open reading frame, the signal peptide and recombinant protein will be transcribed into the same mRNA and translated into the same polypeptide chain. In some embodiments, the first polynucleotide is positioned adjacent to the second polynucleotide in the nucleic acid with no intervening nucleotides between the first and second polynucleotide.

In some embodiments, the methods of the invention comprise transfecting a mammalian cell with the nucleic acid comprising the first polynucleotide and second polynucleotide, culturing the mammalian cell in a cell culture medium under conditions where the recombinant protein is expressed and secreted into the medium, and recovering the recombinant protein from the cell culture medium to obtain a recombinant protein composition. Such method steps are described in detail above. In certain embodiments, following transfection with the nucleic acid, the mammalian cell is cultured in a cell culture medium maintained at a pH of about 6.90 or less for the duration of the production phase of the cell culture as described above. In one embodiment, the mammalian cell is cultured in a cell culture medium maintained at a pH of about 6.70 to about 6.90 for the duration of the production phase of the cell culture. In another embodiment, the mammalian cell is cultured in a cell culture medium maintained at a pH of about 6.70 for the duration of the production phase of the cell culture. In yet another embodiment, the mammalian cell is cultured in a cell culture medium maintained at a pH of about 6.80 for the duration of the production phase of the cell culture. In still another embodiment, the mammalian cell is cultured in a cell culture medium maintained at a pH of about 6.90 for the duration of the production phase of the cell culture.

In certain embodiments of the methods of the invention, the number or amount of alternative splice variant isoforms of a recombinant protein expressed by the mammalian cell is reduced as compared to the number or amount of alternative splice variant isoforms expressed by a mammalian cell comprising a signal peptide encoding-polynucleotide comprising a glycine GGT codon for any glycine residue within the six C-terminal amino acids of a signal peptide. Techniques for detecting and quantitating splice variants are known to those of skill in the art and can include polymerase chain reaction assays, Northern blot analysis, and gel electrophoresis methods.

In certain embodiments, the recombinant protein to be produced according to the methods of the invention is a T-cell engaging molecule, such as a single chain PSMA×CD3 bispecific T-cell engaging molecule. Thus, the present invention also includes isolated nucleic acids encoding a single chain PSMA×CD3 bispecific T-cell engaging molecule, for example a T-cell engaging molecule comprising the amino acid sequence of SEQ ID NO: 1. The term “isolated molecule” (where the molecule is, for example, a protein, a nucleic acid, a polypeptide, or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention can be derived from human sources as well as non-human species. In one embodiment, the isolated nucleic acid encoding a single chain PSMA×CD3 bispecific T-cell engaging molecule comprises the nucleotide sequence of SEQ ID NO: 2 or SEQ ID NO: 3. In another embodiment, the isolated nucleic acid encoding a single chain PSMA×CD3 bispecific T-cell engaging molecule comprises the nucleotide sequence of SEQ ID NO: 4 or SEQ ID NO: 5.

An “isolated nucleic acid” or “isolated polynucleotide” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one embodiment, the nucleic acids are substantially free from contaminating endogenous material. The nucleic acid molecule may have been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 3rd ed., Cold Spring Harbor Laboratory, Cold Spring Harbor, NY (2001)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ production of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The present invention also encompasses vectors, e.g. expression vectors as described above, comprising the nucleic acids encoding the single chain PSMA×CD3 bispecific T-cell engaging molecule as well as host cells or cell lines, particularly mammalian host cells or cell lines, comprising the nucleic acids or expression vectors encoding the single chain PSMA×CD3 bispecific T-cell engaging molecule. In some embodiments, the expression vectors of the invention comprise a nucleic acid comprising the nucleotide sequence of any one of SEQ ID NOs: 2-5. In related embodiments, the present invention provides mammalian host cells transformed with an isolated nucleic acid or expression vector comprising a nucleotide sequence of any one of SEQ ID NOs: 2-5. In certain preferred embodiments, the mammalian host cells are CHO cells. In addition, the present invention includes methods of producing a single chain PSMA×CD3 bispecific T-cell engaging molecule using the expression vectors and transformed host cells or cell lines as described in detail herein. In one embodiment, the method comprises culturing a mammalian host cell transformed with an isolated nucleic acid or expression vector comprising a nucleotide sequence of any one of SEQ ID NOs: 2-5 in a cell culture medium under conditions where the T-cell engaging molecule is expressed, and recovering the T-cell engaging molecule from the culture medium or host cell.

The present invention also includes recombinant protein compositions produced by the methods of the invention. Such recombinant protein compositions have a reduced amount or variety of LMW species of the recombinant protein as compared to the amount or variety of LMW species of the recombinant protein produced by other cell culture methods. In some embodiments, the recombinant protein to be produced by the methods of the invention is an antibody or binding fragment thereof and the recombinant protein composition comprises less than 20% total LMW species of the antibody or binding fragment thereof. In other embodiments, the recombinant protein to be produced by the methods of the invention is a fusion protein and the recombinant protein composition comprises less than 20% total LMW species of the fusion protein. In certain embodiments, the recombinant protein to be produced by the methods of the invention is a T-cell engaging molecule, for example, a single chain T-cell engaging molecule, and the recombinant protein composition comprises less than 20% total LMW species of the T-cell engaging molecule.

In certain related embodiments, the present invention provides a composition comprising a single chain PSMA×CD3 T-cell engaging molecule and one or more LMW species thereof, wherein the composition comprises less than 20% total LMW species of the T-cell engaging molecule, and wherein the T-cell engaging molecule comprises the amino acid sequence of SEQ ID NO: 1. Such compositions of the single chain PSMA×CD3 T-cell engaging molecule may comprise less than 18% total LMW species of the T-cell engaging molecule, for example about 15% or less, about 12% or less, about 10% or less, about 8% or less, or about 6% or less total LMW species of the T-cell engaging molecule. In some embodiments, the compositions of the single chain PSMA×CD3 T-cell engaging molecule comprise about 1% to about 18% total LMW species of the T-cell engaging molecule, such as about 5% to about 15%, about 2% to about 10%, about 1% to about 8%, or about 2% to about 6% total LMW species of the T-cell engaging molecule. As described in Example 2, LMW species of the single chain PSMA×CD3 T-cell engaging molecule exhibited little to no activity in functional assays and thus controlling the amount of such LMW species generated during the production process is important for maintaining the potency of PSMA×CD3 T-cell engaging molecule-containing compositions to an acceptable level. In some embodiments, the LMW species of the single chain PSMA×CD3 T-cell engaging molecule comprises a splice variant isoform of the T-cell engaging molecule. In one such embodiment, the splice variant isoform comprises the amino acid sequence of SEQ ID NO: 23. The amount or level of LMW species of the single chain PSMA×CD3 T-cell engaging molecule in the compositions of the invention can be determined by any of the methods described above for detecting and quantitating these species. In certain embodiments, the amount or level of LMW species in the compositions is determined by a reduced capillary electrophoresis-sodium dodecyl sulfate (rCE-SDS) method. In such a method, LMW species of the single chain PSMA×CD3 T-cell engaging polypeptide elute earlier than the main peak, which corresponds to the full-length single chain PSMA×CD3 T-cell engaging polypeptide (i.e. polypeptide comprising the sequence of SEQ ID NO: 1), and thus correspond to pre-peaks in a rCE-SDS electropherogram. In some embodiments, the rCE-SDS method is conducted as described in Example 1.

The present invention includes pharmaceutical formulations comprising any one of the recombinant protein compositions described herein and one or more pharmaceutically acceptable excipients. For instance, in certain embodiments, the pharmaceutical formulations comprise any one of the single chain PSMA×CD3 T-cell engaging molecule compositions described herein and one or more pharmaceutically acceptable excipients. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical formulation may contain materials for modifying, maintaining or preserving, for example, the pH, osmolarity, viscosity, clarity, color, isotonicity, odor, sterility, stability, rate of dissolution or release, adsorption or penetration of the recombinant protein composition. In such embodiments, suitable materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as glutamate, acetate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring agents, emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol, sorbitol); diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company.

Pharmaceutical formulations comprising the recombinant protein compositions described herein include, but are not limited to, liquid, frozen, and lyophilized formulations. If the pharmaceutical formulation has been lyophilized, the lyophilized material is reconstituted in an appropriate liquid prior to administration. The lyophilized material may be reconstituted in, e.g., bacteriostatic water for injection (BWFI), physiological saline, phosphate buffered saline (PBS), or the same formulation the protein composition had been in prior to lyophilization. Reconstitution volumes will depend on the protein content following lyophilization and the desired concentration of the recombinant protein in the reconstituted solution, but may be from about 0.5 ml to about 5 ml. The solution following reconstitution can be further diluted with a diluent (e.g. saline and/or intravenous solution stabilizer (IVSS)) prior to administration to the patient as appropriate in order to administer the desired doses.

In some embodiments, the pharmaceutical formulations of the invention comprise a recombinant protein composition described herein, a buffer, a stabilizing agent, and optionally a surfactant. Buffers are used to maintain the formulation at physiological pH or at a slightly lower pH, typically within a pH range from about 4.0 to about 6.5. Suitable buffers include, but are not limited to, glutamate, aspartate, acetate, Tris, citrate, histidine, succinate, and phosphate buffers. In certain embodiments, the pharmaceutical formulations comprise a glutamate buffer, particularly L-glutamate buffer. Pharmaceutical formulations comprising a glutamate buffer can have a pH of about 4.0 to about 5.5, a pH of about 4.0 to about 4.4, or a pH of about 4.2 to about 4.8.

A “stabilizing agent” refers to an excipient that stabilizes the native conformation of the recombinant protein and/or prevents or reduces the physical or chemical degradation of the protein. Suitable stabilizing agents include, but are not limited to, polyols (e.g. sorbitol, glycerol, mannitol, xylitol, maltitol, lactitol, erythritol and threitol), sugars (e.g., fructose, glucose, glyceraldehyde, lactose, arabinose, mannose, xylose, ribose, rhamnose, galactose maltose, sucrose, trehalose, sorbose, sucralose, melezitose and raffinose), and amino acids (e.g., glycine, methionine, proline, lysine, arginine, histidine, or glutamic acid). In some embodiments, the pharmaceutical formulation comprises a sugar as a stabilizing agent. In these and other embodiments, the sugar is sucrose.

In certain embodiments, the pharmaceutical formulations comprise a surfactant. The term “surfactant” as used herein refers to a substance that functions to reduce the surface tension of a liquid in which it is dissolved. Surfactants can be included in pharmaceutical formulations for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants that may be incorporated into the pharmaceutical formulations of the invention include both non-ionic and ionic surfactants. Suitable non-ionic surfactants include, but are not limited to, alkyl poly (ethylene oxide), alkyl polyglucosides, such as octyl glucoside and decyl maltoside, fatty alcohols, such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Suitable ionic surfactants include, for example, anionic, cationic and zwitterionic surfactants. Anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate. In certain embodiments, the pharmaceutical formulations comprise a non-ionic surfactant. In one embodiment, the non-ionic surfactant is polysorbate 20. In another embodiment, the non-ionic surfactant is polysorbate 80.

In certain embodiments, a pharmaceutical formulation of the invention comprises about 0.5 mg/mL to about 2 mg/mL of any of the single chain PSMA×CD3 T-cell engaging molecule compositions described herein, about 5 mM to about 20 mM L-glutamic acid, about 0.005% to about 0.015% weight/volume (w/v) polysorbate (e.g. polysorbate 20 or polysorbate 80), and about 7% to about 12% (w/v) sucrose. In other embodiments, the pharmaceutical formulation of the invention comprises about 0.5 mg/mL to about 1.5 mg/mL of any of the single chain PSMA×CD3 T-cell engaging molecule compositions described herein, about 8 mM to about 12 mM L-glutamic acid, about 0.008% to about 0.012% (w/v) polysorbate (e.g. polysorbate 20 or polysorbate 80), and about 8% to about 10% (w/v) sucrose. The pH of these formulations is in the range of about 4.0 to about 4.4 (e.g., pH of about 4.0, about 4.1, about 4.2, about 4.3, or about 4.4). In one particular embodiment, the pharmaceutical formulation comprises about 0.5 mg/mL of a single chain PSMA×CD3 T-cell engaging molecule composition described herein, about 10 mM L-glutamic acid, about 0.010% (w/v) polysorbate 80, and about 9% (w/v) sucrose, wherein the pharmaceutical formulation has a pH of about 4.2.

The pharmaceutical formulations are preferably suitable for parenteral administration. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration. In some embodiments, the pharmaceutical formulation is suitable for intravenous administration. In other embodiments, the pharmaceutical formulation is suitable for subcutaneous administration. Illustrative pharmaceutical forms suitable for parenteral administration include sterile aqueous solutions or dispersions and sterile powders for the extemporaneous preparation of sterile injectable solutions or dispersions. Preferably, the pharmaceutical formulation is sterile and is sufficiently fluid to allow for delivery via a syringe or other injection device (i.e., the formulation is not excessively viscous so as to prevent passage through a syringe or other injection device). Sterilization can be accomplished by filtration through sterile filtration membranes. When the composition is lyophilized, sterilization using this filtration method may be conducted either prior to or following lyophilization and reconstitution. Pharmaceutical formulations for parenteral administration can be stored in lyophilized form or in a solution. Parenteral formulations can be placed into a container having a sterile access port, for example, an intravenous solution bag or vial having a stopper pierceable by a hypodermic injection needle. Parenteral formulations can also be stored in syringes, autoinjector devices, or pen injection devices or cartridges adapted for use with such injection devices.

Parenteral, subcutaneous, or intravenous administration can be performed by injection (e.g. using a needle and a syringe) or by infusion (e.g. via a catheter and a pump system). It is envisaged that in some embodiments the administration according to the present invention is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

The pharmaceutical formulations described above can be filled into vials, syringes, autoinjectors, or other containers or delivery devices and optionally packaged into kits with instructions for use (e.g. prescribing information containing instructions for using the pharmaceutical formulations for treating, preventing, or reducing the occurrence of a disease, disorder, or condition, e.g. cancer) to prepare pharmaceutical products. The pharmaceutical formulation may be provided as a solution, suspension, gel, emulsion, solid, crystal, or as a dehydrated or lyophilized powder. In embodiments in which the pharmaceutical formulation is provided as a lyophilized powder, the kit may also comprise diluents (e.g. sterile water for injection, saline, phosphate-buffered saline, formulation buffer) necessary to reconstitute the pharmaceutical formulation as well as instructions for preparing the formulation for administration. In certain embodiments in which the pharmaceutical formulation is intended to be administered intravenously, the kits may further comprise one or more vials of intravenous solution stabilizer (IVSS) and instructions for using the IVSS for pre-treatment of IV bags prior to dilution of the pharmaceutical formulation for delivery to the patient. IVSS does not contain an active pharmaceutical ingredient and is typically a buffered, preservative-free solution. In one embodiment, IVSS comprises citric acid (e.g. 20-30 mM), lysine hydrochloride (e.g. 1-3 M), and polysorbate 80 (0.05%-0.15% (w/v)) at pH 7.0. In a particular embodiment, IVSS comprises 25 mM citric acid, 1.25 M lysine hydrochloride, and 0.1% (w/v) polysorbate 80 at pH 7.0.

The recombinant protein compositions described herein and pharmaceutical formulations comprising such compositions can be used to treat, prevent or reduce the occurrence of a disease, disorder, or condition in a patient in need thereof. The term “treatment” or “treat” as used herein refers to the application or administration of the recombinant protein compositions or pharmaceutical formulations comprising the compositions to a patient who has or is diagnosed with a disease, disorder, or condition (e.g. cancer), has a symptom of the disease, disorder, or condition, is at risk of developing the disease, disorder, or condition, or has a predisposition to the disease, disorder, or condition for the purpose of curing, healing, alleviating, relieving, altering, ameliorating, or improving the disease, disorder, or condition, one or more symptoms of the disease, disorder, or condition, the risk of developing the disease, disorder, or condition, or predisposition toward the disease, disorder, or condition. The term “treatment” encompasses any improvement of the disease in the patient, including the slowing or stopping of the progression of disease in the patient, a decrease in the number or severity of the symptoms of the disease, or an increase in frequency or duration of periods where the patient is free from the symptoms of the disease. The term “patient” includes human patients.

In certain embodiments, the single chain PSMA×CD3 T-cell engaging molecule compositions described herein and pharmaceutical formulations comprising such compositions can be used to treat a PSMA-expressing cancer in a patient in need thereof. Accordingly, the present invention includes methods for treating a PSMA-expressing cancer in a patient in need thereof comprising administering to the patient any of the single chain PSMA×CD3 T-cell engaging molecule compositions or pharmaceutical formulations comprising the compositions described herein. In some embodiments, the present invention provides single chain PSMA×CD3 T-cell engaging molecule compositions or pharmaceutical formulations comprising the compositions described herein for use in a method for treating a PSMA-expressing cancer in a patient in need thereof. In other embodiments, the present invention encompasses the use of single chain PSMA×CD3 T-cell engaging molecule compositions or pharmaceutical formulations comprising the compositions described herein in the preparation of a medicament for treating a PSMA-expressing cancer in a patient in need thereof.

The term “cancer” refers to various conditions caused by the abnormal, uncontrolled growth of cells and includes neoplasms, primary tumors, secondary tumors and other metastatic lesions. The term “cancer” encompasses various cancerous conditions regardless of stage, grade, invasiveness, aggressiveness, or tissue type. PSMA-expressing cancer refers to cancerous conditions in which the neoplasms, primary tumors, secondary tumors or other metastatic lesions contain cells expressing a detectable level of PSMA protein on their surface, by for example histological or radiological means (PSMA PET scan). Cancer can be detected in a number of ways including, but not limited to, the presence of a tumor in a tissue as detected by clinical or radiological means, detection of cancerous or abnormal cells in a biological sample (e.g. tissue biopsy), detection of a biomarker indicative of a cancer or a pre-cancerous condition (e.g. prostate specific antigen (PSA)), or detection of a genotype indicative of cancer or the risk of developing cancer. PSMA-expressing cancers include, but are not limited to, prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, and melanoma. In some embodiments, the PSMA-expressing cancer is prostate cancer. The prostate cancer may be castration-resistant prostate cancer (prostate cancer that is resistant to androgen deprivation therapy). In these and other embodiments, the prostate cancer is metastatic prostate cancer, particularly metastatic castration-resistant prostate cancer.

The following examples, including the experiments conducted and the results achieved, are provided for illustrative purposes only and are not to be construed as limiting the scope of the appended claims.

### EXAMPLES

### Example 1. Reduction of Low Molecular Weight Species Variants of a Recombinant Polypeptide Through pH Control of Production Culture

Recombinant production of proteins can result in the generation of variants of the protein, some of which may have undesirable properties or characteristics, such as reduced functional activity. An example of such a product-related impurity is low molecular weight (LMW) species of the protein. LMW species can include various truncated forms of the protein arising from: cellular expression of truncated forms, such as alternative mRNA splice variants; enzymatic clipping of the expressed protein; or incomplete assembly of the polypeptide chains in the case of multi-chain proteins. Because LMW species of a protein can impact the purity and overall activity of drug substance, control of the formation of LMW species during protein production is important. This example describes the impact of production cell culture pH on the levels of LMW species of a single-chain bispecific T-cell engaging molecule.

Bispecific T-cell engaging molecules are designed to direct T lymphocyte effector cells towards target cancer cells. The proximity of the T-cell to the target cancer cell induced by the bispecific T-cell engaging molecule triggers T-cell activation resulting in the T-cell-mediated cytotoxicity of the target cancer cell. A half-life extended bispecific T-cell engager that binds prostate-specific membrane antigen (PSMA) on cancer cells and cluster of differentiation 3 epsilon (CD3E) on T-cells was designed as a single-chain polypeptide comprising a single-chain variable fragment (scFv) domain with binding specificity for human PSMA, a scFv domain with binding specificity for human CD38, and a single-chain Fc domain. The amino acid sequence of the PSMA×CD3 T-cell engager polypeptide is set forth in SEQ ID NO: 1. A nucleic acid comprising the nucleotide sequence of SEQ ID NO: 2 encoding the PSMA×CD3 T-cell engager polypeptide was cloned into a mammalian expression vector and stably transfected into Chinese hamster ovary (CHO) cells.

After thawing, the T-cell engager polypeptide-producing CHO cell line was cultured in a serum-free selective growth medium in a series of shake flasks followed by culturing in a chemically defined selective growth medium in two-stage 3 L shake flasks (N-3, N-2). Cultures were incubated at a temperature of 36.0° C., 5.0% CO₂ and expanded until sufficient cell mass was obtained to inoculate the N-1 and production (N) bioreactors. Culture was transferred from the N-2 shake flask to a N-1 3 L bioreactor (working volume of 1.5 L). The N-1 bioreactor was operated in batch mode for 4 days with the following parameters: temperature at 36.0° C., pH 6.90, dissolved oxygen (DO) at 64 mm Hg, and agitation at 350 RPM. The 3 L production (N) bioreactor (working volume of approximately 1.5 L) was seeded at an initial viable cell density of about 10×10⁵ cells/mL and run in batch mode from day 0 to day 3 and then in perfusion mode from day 3 to day 15 using an alternating tangential flow (ATF) filtration system. From day 3 to day 15, the cell culture was continuously fed with a serum-free chemically defined perfusion medium at an initial rate of 0.50 bioreactor volume/day that was increased to 1.0 bioreactor volume/day by day 8. The production bioreactor was operated at the following parameters: temperature at 36.0° C. initially and decreased to 32.5° C. on day 7, DO at 64 mm Hg, and agitation at 350 RPM. To evaluate the effect of production culture pH on the generation of LMW species of the T-cell engager polypeptide, the pH setpoint of the production bioreactor was evaluated at 6.70, 6.80, 6.90, and 7.10. Glucose solution was fed to the bioreactor as needed to maintain a glucose concentration of ≥4.0 g/L. The bioreactor was harvested by switching the filter in the ATF filtration system to a microfilter to allow the T-cell engager polypeptide to pass through the filter into the permeate and retain the cells and cell debris in the bioreactor. The permeate from the microfilter was collected to obtain the harvested cell culture fluid (HCCF). Daily samples were taken from the production bioreactor to assess the culture. Viable cell density (VCD) and cell viability were determined using a Cedex HiRes cell culture analyzer (Roche Diagnostics Corporation, Indianapolis, IN).

LMW species of the T-cell engager polypeptide were measured in the HCCF collected from the bioreactors operated at the different pH set points using a reduced capillary electrophoresis-sodium dodecyl sulfate (rCE-SDS) method, which separates polypeptides based on differences in their hydrodynamic size under reducing and denaturing conditions. Samples of the HCCF were purified by protein A chromatography to separate the T-cell engager polypeptide from cell debris and other matrix components. A portion of the purified material was then mixed with reducing sample buffer containing sodium dodecyl sulfate (SDS) and β-mercaptoethanol. Samples were incubated at 70° ° C. for ten minutes prior to electrokinetic injection into a bare-fused silica capillary filled with SDS gel buffer (Beckman Coulter, Brea, CA) at 25° C. Polypeptides were detected by a photodiode array detector as they passed through a UV detection window. UV absorbance was monitored at 220 nm. Quantitation of the LMW species was based on the relative area percentage of peaks eluting earlier than the main peak, which corresponded to the full-length T-cell engager polypeptide.

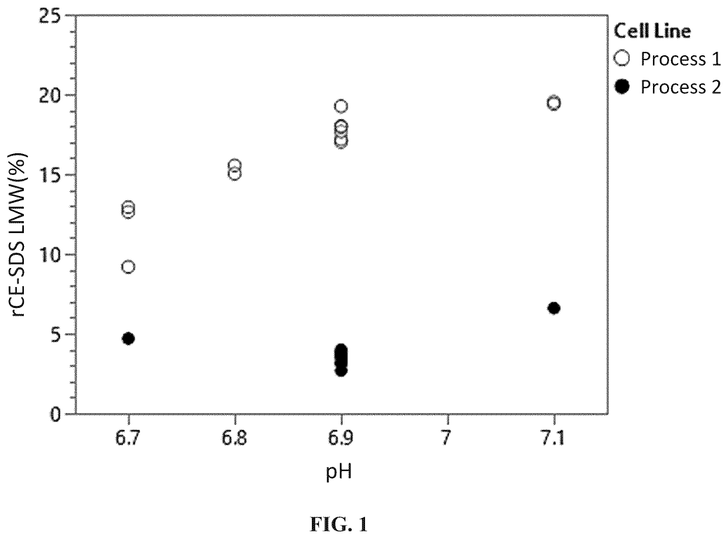

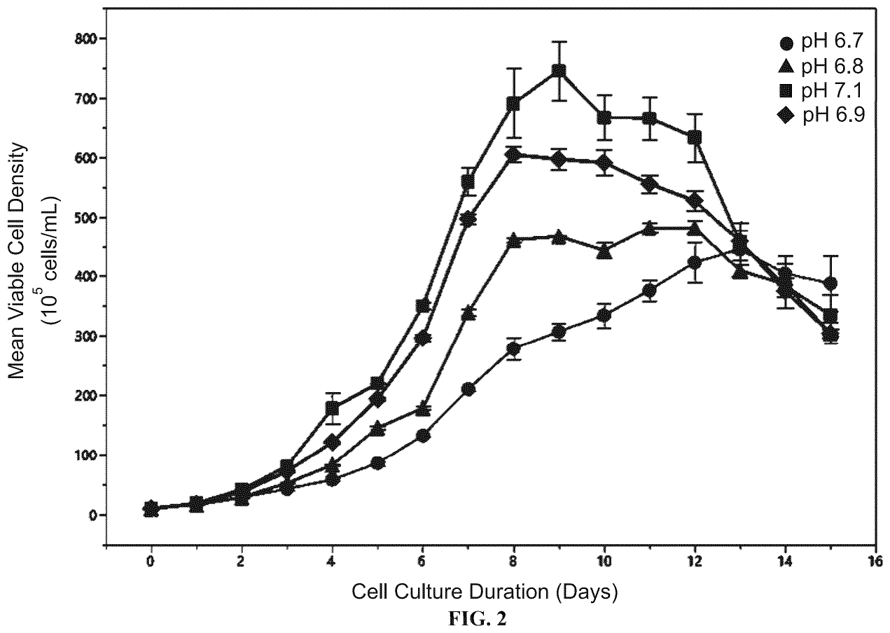

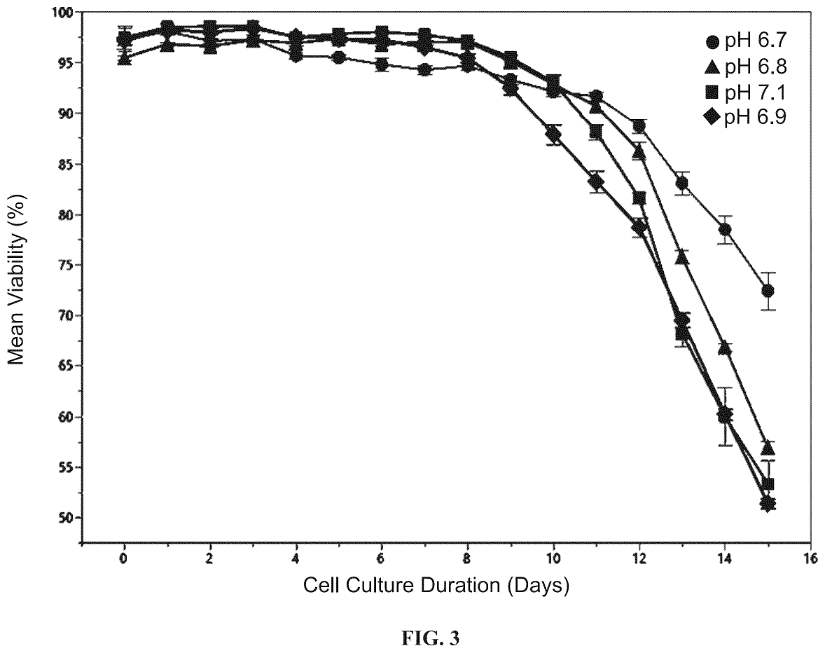

The results of these experiments show that decreasing the production bioreactor pH setpoint led to a reduction in total LMW species of the T-cell engager polypeptide in the HCCF (FIG. 1, process 1 cell line). The total LMW species in HCCF were reduced from an average of 19.5% to an average of 11.6% when the production bioreactor pH setpoint was adjusted from 7.10 to 6.70 (Table 1). Over the pH range of 6.70 to 7.10, the relationship between pH and LMW species could be modeled with a quadratic best-fit curve and statistical analysis showed a strong correlation between production bioreactor pH and total LMW species, with a coefficient of determination of R²=0.9 and an adjusted R²=0.89. One-way ANOVA and Tukey-Kramer analysis showed statistically significant differences in percentage of LMW species between the pH 7.10 condition and the pH 6.80 and 6.70 conditions as well as between the pH 6.80 condition and the pH 6.70 condition (Table 1). In addition, production bioreactor pH impacted cell culture growth (VCD) and viability (percentage of viable cells), with pH 6.70 bioreactors having reduced VCD through culture day 12 relative to bioreactors operated at higher pH values (FIG. 2). However, production bioreactors run at a pH setpoint of 6.70 had a higher viability at the end of the 15-day culture period relative to the other bioreactors run at higher pH setpoints (FIG. 3).

TABLE 1
Effect of Production Bioreactor pH on Total LMW Species in HCCF

| Production | Number of | | |
|---|---|---|---|
| Bioreactor | Production | % Total LMW Species of T-cell Engager Polypeptide | |
| setpoint pH | Bioreactor Runs | Mean | Standard Error of the Mean |
| 6.70 | 3 | 11.60 | 0.60 |
| 6.80 | 2 | 15.30 | 0.74 |
| 6.90 | 8 | 17.86 | 0.37 |
| 7.10 | 2 | 19.47 | 0.74 |

In sum, the experiments described in this Example demonstrate that reducing the pH setpoint of the production bioreactor results in reduced formation of LMW species of a PSMA×CD3 bispecific T-cell engager polypeptide during the cell culture production process.

### Example 2. Low Molecular Weight Species Variants Affect Functional Activity

To further characterize the properties of the LMW species of the PSMA×CD3 bispecific T-cell engager polypeptide and their impact on the functional activity of drug substance, a cation exchange (CEX) chromatography method was developed to isolate the LMW species. Generally, CEX chromatography separates proteins based primarily on the heterogeneity of surface charge. Peak elution in this method is a function of net surface charge with negatively charged species (more acidic species) eluting earlier and positively charged species (more basic species) eluting later.

HCCF obtained from cells expressing the PSMA×CD3 bispecific T-cell engager polypeptide described in Example 1 was partially purified by protein A chromatography and then loaded onto a CEX chromatography column that utilized Capto-SP ImpRes® cation exchange chromatography resin (GE Healthcare Bio-Science, Marlborough, MA). Mobile phase A contained 100 mM acetate, 215 mM sodium chloride at pH 4.5 and mobile phase B consisted of 100 mM acetate, 350 mM sodium chloride at pH 4.5. Proteins were separated using a linear salt gradient generated with 0% to 80% mobile phase B over 18 column volumes (CV). The eluent was monitored by UV absorbance at 280 nm. The mobile phase was applied to the column at a flow rate of 150 cm/hr. A representative chromatogram is shown in FIG. 4. As shown in the figure, the peak enriched in the LMW species of the T-cell engager polypeptide elutes later than the full-length polypeptide (represented by the Main Peak eluting at about 20 CV) and thus the LMW species are more positively charged than the full-length polypeptide (i.e. are basic species of the T-cell engager polypeptide). The post-peak enriched in the LMW species was collected, diluted 1:6 with purified water, and re-loaded onto the CEX column and subject to a second cycle of separation. The post-peak enriched in the LMW species was collected from this second cycle, diluted 1:6 with purified water, and re-loaded on the CEX column and subject to a third cycle of separation. The post-peak enriched in the LMW species was collected a final time and dialyzed into a formulation buffer (10 mM glutamate, 9% (w/v) sucrose, pH 4.2) and passed through a membrane filter (Mustang E membrane, Pall Corporation, Port Washington, NY) to remove endotoxins. This fraction enriched in the LMW species of the T-cell engager polypeptide was used to spike drug substance containing the T-cell engager polypeptide with specific amounts (25%, 50%, or 75%) of the LMW species. Analytical testing of the spiked drug substance samples by the rCE-SDS method described in Example 1 was conducted to verify the amounts of LMW species in the samples (data not shown).

The drug substance samples spiked with the different amounts of the LMW species were tested for activity in a cell-based potency assay and binding assay. For the cell-based potency assay, a human CD4″ T cell effector cell line expressing a luciferase reporter driven by nuclear factor of activated T cells response element (NFAT-RE) (Jurkat NFAT-RE Luc cells; catalog #J1621, Promega, Madison, WI) and C4-2B cells, a prostate cancer cell line naturally expressing human PSMA, were used. The PSMA×CD3 bispecific T-cell engager polypeptide binds to PSMA on the C4-2B cells and to CD3 on the Jurkat NFAT-RE Luc cells thereby bringing the T-cells into proximity with the C4-2B target cells and activating the T-cells resulting in NFAT-RE-mediated luminescence. Cells were incubated for 3 to 6 hours with the different drug substance samples and then luciferase substrate was added. T-cell activation was assessed by measuring the luminescence signal with a plate reader. The activity in the cell-based potency assay for each of the drug substance samples spiked with different amounts of the LMW species was normalized to the activity of the Reference Standard for the T-cell engager polypeptide and reported as a percent relative potency.

The binding assay utilized a homogeneous proximity-based format to measure the ability of the PSMA×CD3 bispecific T-cell engager polypeptide to bind to both a histidine-tagged prostate specific membrane antigen (PSMA his) and a biotinylated cluster of differentiation 3 epsilon antigen (CD38-biotin). Specifically, varying concentrations of the PSMA×CD3 bispecific T-cell engager polypeptide were incubated with fixed concentrations of both CD3ε-biotin and PSMA-his and donor and acceptor beads. The donor beads were coated with a hydrogel that contains phthalocyanine, a photosensitizer, and streptavidin. The acceptor beads were coated with a hydrogel that contains thioxene derivatives and nickel chelate. When the PSMA×CD3 bispecific T-cell engager polypeptide binds to CD38-biotin and PSMA-his, the donor beads, coated with streptavidin, will bind to the biotinylated CD38, and the acceptor beads, coated with nickel chelate, will bind to the PSMA-his, which will bring the beads into proximity. When a laser is applied to this complex, ambient oxygen is converted to singlet oxygen by the donor bead. If the beads are in proximity, a series of chemical reactions in the acceptor bead is induced by the singlet oxygen, resulting in light production (luminescence), which is measured by a plate reader. This binding assay measured the dose dependent increase in signal observed when the PSMA×CD3 bispecific T-cell engager polypeptide bound to CD38-biotin and PSMA-his. Test drug substance sample activity was determined by comparing the test sample response to the response obtained for the PSMA×CD3 bispecific T-cell engager polypeptide Reference Standard using a 5-point parallel line analysis format and reported as a percent relative potency.

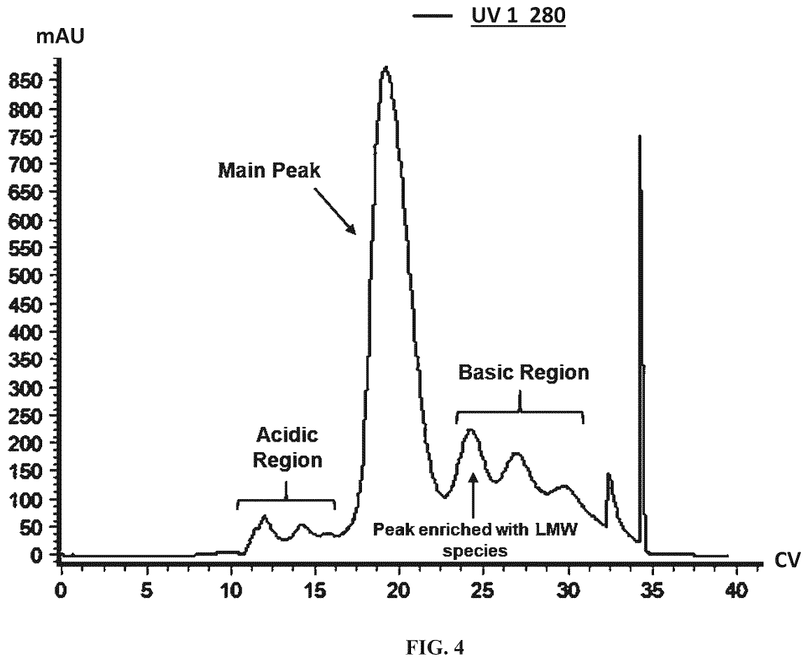

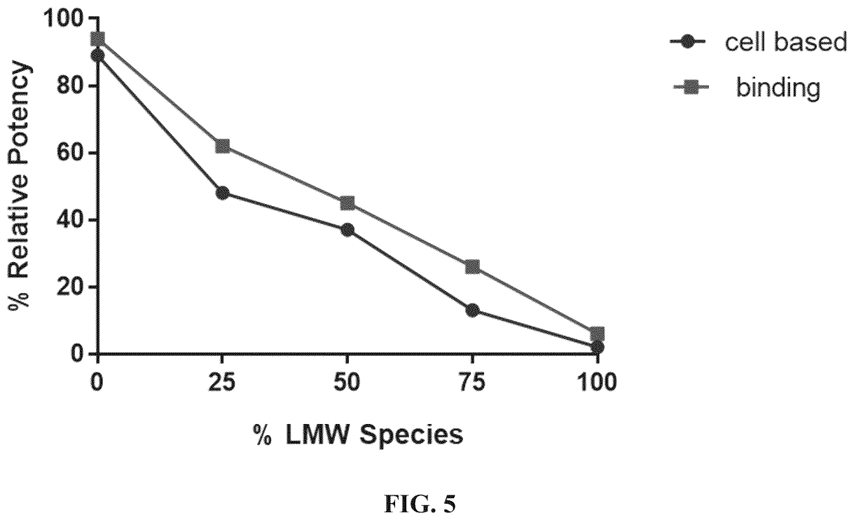

As shown in FIG. 5, the results of the activity assays reveal that the potency of the drug substance in both assays decreases with increasing amounts of the LMW species present in the drug substance. The sample enriched for LMW species of the T-cell engager polypeptide (labeled as 100% LMW species in the figure) exhibited very little activity in both assays. When the drug substance contained even 25% LMW species, the potency of the drug substance in the cell-based activity assay was reduced by nearly 50%. Because the LMW species of the T-cell engager polypeptide are inactive product variants, the results of the experiments described in this example highlight the importance of controlling the generation of these LMW species during the production process, for example using the method described in Example 1.

### Example 3. Reduction of Low Molecular Weight Species Variants of a Recombinant Polypeptide Via Codon Optimization to Prevent Alternative RNA Splicing

In eukaryotic cells, genomic DNA is first transcribed into pre-messenger RNA (mRNA), which contains both protein coding sequences (exons) and non-protein coding sequences (introns). Subsequently, a spliceosome, an RNA splicing complex, removes introns and joins exons together to create the final mature mRNA sequence that encodes for the desired protein. The spliceosome recognizes donor, acceptor, and branchpoint sites within the intron/exon junctions of the pre-mRNA. For recombinant protein production, introns are typically not included in the nucleic acid sequence encoding the protein of interest. Rather, complementary DNA (cDNA), which is the DNA copy of the desired mature mRNA sequence, is used. However, the presence of near consensus splice donor and acceptor sites within the cDNA encoding for recombinant proteins can sometimes trigger unintended alternative splicing events. This in turn can result in modifications to the protein amino acid sequence including overhangs, deletions, and insertions. Furthermore, it can be challenging to predict when these alternative splicing events will occur based on sequence analysis alone because the presence of a splice donor or acceptor site does not necessarily mean splicing will occur and splicing events can depend on nucleotide sequences flanking the donor and acceptor sites, such as the genomic context around the site where the cDNA encoding the recombinant protein integrates into the genome of the host cell (see, e.g., Zheng et al., RNA, Vol. 11: 1777-1787, 2005; Rotival et al., Nat. Commun., Vol. 10, 1671, 2019).

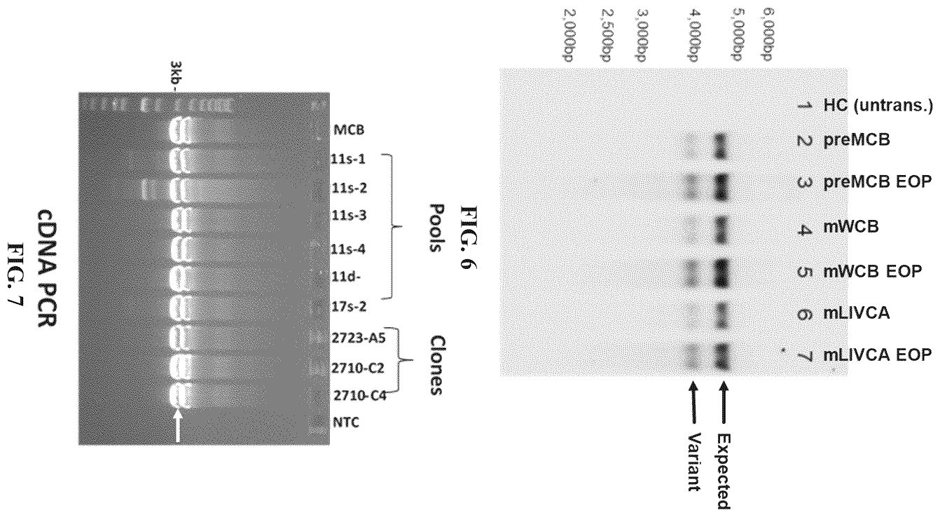

Elevated levels of LMW species of the PSMA×CD3 bispecific T-cell engager polypeptide (e.g. >20%) were observed in the HCCF from the cell line described in Example 1 and three other CHO cell clones stably transfected with the nucleic acid comprising the nucleotide sequence of SEQ ID NO: 2. Genetic characterization revealed the presence of a single truncated transcript variant that was consistent over time and independent of cell age (FIG. 6). Polymerase chain reaction (PCR) analysis with primers flanking the coding sequence was performed on genomic DNA and cDNA (prepared from RNA) isolated from different clones and pools. The results of this analysis showed that the variant was detected in all clones and pools from the cDNA PCR analysis (FIG. 7) but not the genomic DNA PCR analysis (data not shown), thereby confirming that the variant was a transcript variant resulting from alternative splicing.

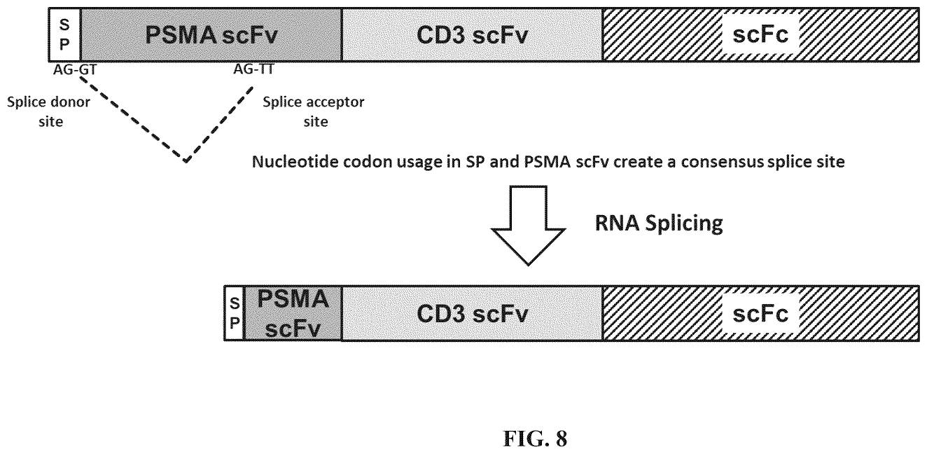

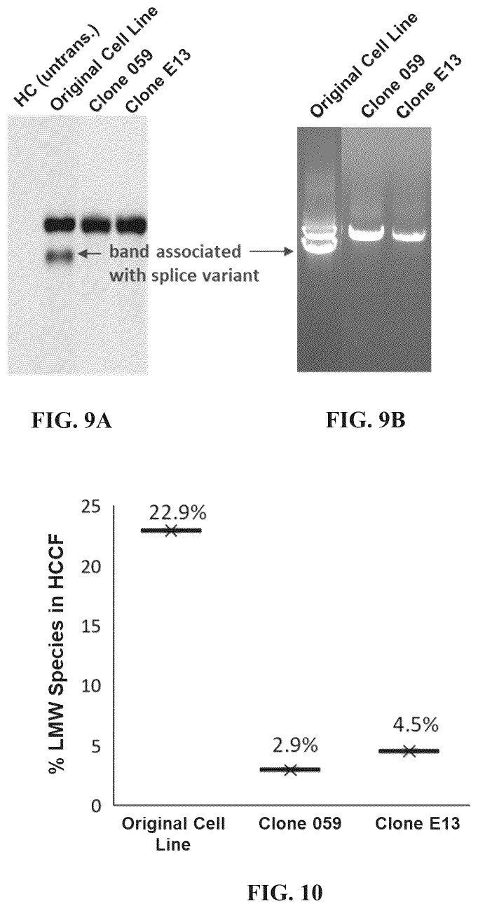

It was hypothesized that alternative splicing using a combination of a strong splice donor site (GAG|gtgcg) in the kappa variable 1 signal peptide at nucleotides 53-60 of SEQ ID NO: 2 and a splice acceptor site (ctatttcatcAG|TT) in the PSMA scFv domain at nucleotides 695-708 of SEQ ID NO: 2 was a likely mechanism for the generation of this transcript variant resulting in a deletion of 651 nucleotides (FIG. 8). To test this hypothesis, the consensus splice donor site in the kappa variable 1 signal peptide nucleotide sequence was eliminated by replacing the glycine codon GGT at nucleotides 55-57 with glycine codon GGG. In addition, the consensus splice acceptor site in the PSMA scFv nucleotide sequence was weakened by replacing serine codon TCA at nucleotides 700-703 and 704-706 with serine codon TCC (A|GTT to C|GTT). The amino acid sequence of the encoded PSMA×CD3 T-cell engager polypeptide was not affected by these codon changes. The modified nucleic acid sequence (SEQ ID NO: 4) was cloned into a mammalian expression vector and stably transfected into CHO cells. As shown in FIGS. 9A and 9B, these codon modifications in the optimized nucleic acid sequence set forth in SEQ ID NO: 4 eliminated the generation of the shorter transcript variant as the variant was not detectable by Northern blot or RT-PCR analysis of RNA isolated from clones expressing the modified nucleic acid sequence. HCCF obtained from cells expressing the modified nucleic acid sequence was analyzed by the rCE-SDS method described in Example 1 to quantify the amount of LMW species. The percent of LMW species present in HCCF isolated from the cells expressing the modified nucleic acid sequence was significantly reduced to below 5% from the 23% LMW species observed in HCCF from the original cell line (FIG. 10). These results suggest that the elevated levels of LMW species observed in the original cell lines were due to the production of truncated variants of the polypeptide resulting from an alternative transcript variant. Because the strong splice donor site in the kappa variable 1 signal peptide sequence is naturally occurring, the codon modification approach described in this example to replace the GGT glycine codon at the 3′ end of the sequence with the GGG glycine codon could be used in the production of any recombinant protein products using this signal peptide or a signal peptide with a glycine residue within the six carboxy-terminal amino acids to avoid alternative splicing.

To determine whether pH of the production culture also affected the percentage of LMW species in this second cell line expressing the modified nucleic acid sequence encoding the PSMA×CD3 bispecific T-cell engager polypeptide, the cell line was cultured and expanded as described in Example 1. The pH setpoint of the production bioreactor was evaluated at 6.70, 6.90, and 7.10. The operating parameters of the production bioreactor and harvest process were the same as that described in Example 1. Although the percent of LMW species in the HCCF obtained from this second cell line (process 2 cell line) is much lower than the LMW species in HCCF obtained from the process 1 cell line, which contained a different nucleic acid encoding the T-cell engager polypeptide, reduction of the pH setpoint in the production bioreactor appeared to further reduce the total LMW species present. See FIG. 1, process 2 cell line.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize or be able to ascertain using no more than routine experimentation, many equivalents to the specific embodiments of the invention described herein. Such equivalents are intended to be encompassed by the following claims.

TABLE 2
Sequence Listing

| SEQ ID | | |
|---|---|---|
| NO: | Description | Sequence |
| 1 | PSMA x CD3 T-cell | QVQLVESGGGLVKPGESLRLSCAASGFTFSDYYMYWVRQ |
| | engager polypeptide | APGKCLEWVAIISDGGYYTYYSDIIKGRFTISRDNAKNSLYL |
| | amino acid sequence | QMNSLKAEDTAVYYCARGFPLLRHGAMDYWGQGTLVTV |
| | | SSGGGGSGGGGSGGGGSDIQMTQSPSSLSASVGDRVTITCK |
| | | ASQNVDTNVAWYQQKPGQAPKSLIYSASYVYWDVPSRFS |
| | | GSASGTDFTLTISSVQSEDFATYYCQQYDQQLITFGCGTKL |
| | | EIKSGGGGSEVQLVESGGGLVQPGGSLKLSCAASGFTFNKY |
| | | AMNWVRQAPGKGLEWVARIRSKYNNYATYYADSVKDRF |
| | | TISRDDSKNTAYLQMNNLKTEDTAVYYCVRHGNFGNSYIS |
| | | YWAYWGQGTLVTVSSGGGGSGGGGSGGGGSQTVVTQEPS |
| | | LTVSPGGTVTLTCGSSTGAVTSGNYPNWVQQKPGQAPRGL |
| | | IGGTKFLAPGTPARFSGSLLGGKAALTLSGVQPEDEAEYYC |
| | | VLWYSNRWVFGGGTKLTVLGGGGDKTHTCPPCPAPELLG |
| | | GPSVFLFPPKPKDTLMISRTPEVTCVVVDVSHEDPEVKFNW |
| | | YVDGVEVHNAKTKPCEEQYGSTYRCVSVLTVLHQDWLNG |
| | | KEYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGKGGGGSGGGGSGGGGSGGGGSGGGGSGGG |
| | | GSDKTHTCPPCPAPELLGGPSVFLFPPKPKDTLMISRTPEVT |
| | | CVVVDVSHEDPEVKFNWYVDGVEVHNAKTKPCEEQYGST |
| | | YRCVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 2 | PSMA x CD3 T-cell | ATGGACATGAGGGTGCCCGCTCAGCTCCTGGGGCTCCTG |
| | engager polypeptide- | CTGCTGTGGCTGAGAGGTGCGCGCTGTCAGGTGCAGCTC |
| | encoding nucleic acid | GTGGAATCCGGTGGCGGCTTGGTGAAACCTGGCGAGTCA |
| | #1 | CTGAGATTGTCATGCGCTGCATCAGGTTTTACATTCAGT |
| | | GATTACTATATGTACTGGGTACGGCAGGCACCTGGAAAA |
| | | TGCCTTGAGTGGGTCGCTATTATCAGTGACGGCGGGTAT |
| | | TATACCTATTACTCCGATATCATTAAAGGAAGGTTTACC |
| | | ATCAGCCGAGATAACGCCAAAAACTCCCTTTACCTTCAG |
| | | ATGAACAGTCTTAAAGCTGAGGACACTGCTGTTTACTAT |
| | | TGTGCTAGAGGGTTCCCCCTGCTGCGTCACGGCGCAATG |
| | | GATTACTGGGGCCAGGGCACCCTTGTAACCGTAAGCAGT |
| | | GGTGGGGGGGGTAGTGGTGGTGGTGGCTCTGGCGGTGG |
| | | AGGATCAGACATCCAGATGACCCAAAGCCCCTCTAGTCT |
| | | CAGCGCAAGTGTAGGCGACCGGGTAACCATTACATGTA |
| | | AGGCATCACAGAATGTGGACACAAATGTTGCTTGGTATC |
| | | AGCAGAAGCCAGGACAGGCTCCTAAGTCACTTATCTACA |
| | | GTGCAAGTTATGTGTATTGGGACGTTCCTTCCCGCTTTTC |
| | | TGGTTCCGCTAGTGGAACTGACTTCACTTTGACTATTTCA |
| | | TCAGTTCAGTCTGAGGACTTCGCCACATATTACTGCCAA |
| | | CAATATGATCAGCAGCTCATTACCTTTGGCTGTGGCACA |
| | | AAGCTGGAAATCAAGTCCGGAGGTGGTGGATCCGAGGT |
| | | GCAGCTGGTCGAGTCTGGAGGAGGATTGGTGCAGCCTG |
| | | GAGGGTCATTGAAACTCTCATGTGCAGCCTCTGGATTCA |
| | | CCTTCAATAAGTACGCCATGAACTGGGTCCGCCAGGCTC |
| | | CAGGAAAGGGTTTGGAATGGGTTGCTCGCATCAGAAGT |
| | | AAATATAATAATTATGCAACATATTATGCCGATTCAGTG |
| | | AAAGACAGGTTCACCATCTCCAGAGATGATTCAAAAAA |
| | | CACTGCCTATCTACAAATGAACAACTTGAAGACTGAGGA |
| | | CACTGCCGTGTACTACTGTGTGAGACATGGGAACTTCGG |
| | | TAATAGCTACATCTCCTACTGGGCTTACTGGGGCCAAGG |
| | | GACTCTGGTCACCGTGTCCTCAGGTGGTGGTGGTTCTGG |
| | | CGGCGGCGGCTCCGGTGGTGGTGGTTCTCAGACTGTTGT |
| | | GACTCAGGAACCTTCACTCACCGTATCACCTGGTGGAAC |
| | | AGTCACACTCACTTGTGGCTCCTCGACTGGGGCTGTTAC |
| | | ATCTGGCAACTACCCAAACTGGGTCCAACAAAAACCAG |
| | | GTCAGGCACCCCGTGGTCTAATCGGTGGGACTAAGTTCC |
| | | TCGCCCCCGGTACTCCTGCCAGATTCTCAGGCTCCCTGCT |
| | | TGGAGGCAAGGCTGCCCTCACCCTCTCAGGGGTACAGCC |
| | | AGAGGATGAGGCGGAATATTACTGTGTTCTATGGTACAG |
| | | CAACCGCTGGGTGTTCGGTGGAGGAACCAAACTGACTGT |
| | | CCTAGGCGGTGGTGGAGACAAAACTCACACATGCCCAC |
| | | CGTGCCCAGCACCTGAACTCCTGGGGGGACCGTCAGTCT |
| | | TCCTCTTCCCCCCAAAACCCAAGGACACCCTCATGATCT |
| | | CCCGGACCCCTGAGGTCACATGCGTGGTGGTGGACGTGA |
| | | GCCACGAAGACCCTGAGGTCAAGTTCAACTGGTACGTGG |
| | | ACGGCGTGGAGGTGCATAATGCCAAGACAAAGCCGTGC |
| | | GAGGAGCAGTACGGCAGCACGTACCGTTGCGTCAGCGT |
| | | CCTCACCGTCCTGCACCAGGACTGGCTGAATGGCAAGGA |
| | | GTACAAGTGCAAGGTGTCCAACAAAGCCCTCCCAGCCCC |
| | | CATCGAGAAAACCATCTCCAAAGCCAAAGGGCAGCCCC |
| | | GAGAACCACAGGTGTACACCCTGCCCCCATCCCGGGAG |
| | | GAGATGACCAAGAACCAGGTCAGCCTGACCTGCCTGGTC |
| | | AAAGGCTTCTATCCCAGCGACATCGCCGTGGAGTGGGAG |
| | | AGCAATGGGCAGCCGGAGAACAACTACAAGACCACGCC |
| | | TCCCGTGCTGGACTCCGACGGCTCCTTCTTCCTCTATAGC |
| | | AAGCTCACCGTGGACAAGAGCAGGTGGCAGCAGGGGAA |
| | | CGTCTTCTCATGCTCCGTGATGCATGAGGCTCTGCACAA |
| | | CCACTACACGCAGAAGAGCCTCTCCCTGTCTCCGGGTAA |
| | | AGGTGGAGGAGGTAGCGGAGGAGGCGGCTCCGGAGGGG |
| | | GCGGATCTGGCGGCGGAGGAAGCGGTGGGGGCGGCTCC |
| | | GGAGGGGGCGGCTCTGACAAGACCCACACATGTCCTCC |
| | | GTGCCCCGCACCCGAGCTGTTGGGAGGACCCTCGGTGTT |
| | | TTTGTTTCCTCCCAAGCCAAAGGACACGTTGATGATTTC |
| | | GCGCACTCCAGAGGTCACGTGTGTAGTCGTGGACGTGTC |
| | | ACATGAGGACCCCGAGGTAAAGTTCAATTGGTATGTGGA |
| | | CGGAGTCGAGGTCCATAACGCCAAAACAAAGCCGTGCG |
| | | AAGAACAGTACGGGTCAACGTATAGGTGCGTCAGCGTC |
| | | CTCACTGTGCTGCACCAAGACTGGCTCAATGGTAAAGAA |
| | | TACAAGTGCAAGGTGTCGAACAAGGCCCTCCCTGCCCCT |
| | | ATCGAGAAAACCATCTCCAAAGCGAAGGGGCAGCCGCG |
| | | AGAACCCCAAGTCTACACGCTGCCGCCCTCGCGGGAGG |
| | | AAATGACCAAAAACCAGGTGTCGCTTACGTGTCTTGTGA |
| | | AAGGGTTCTATCCATCAGATATCGCGGTCGAGTGGGAGT |
| | | CGAACGGCCAGCCCGAAAACAATTACAAAACAACACCT |
| | | CCGGTCCTCGATTCAGATGGAAGCTTCTTCTTGTATTCGA |
| | | AGCTGACCGTCGATAAGTCAAGGTGGCAACAGGGAAAT |
| | | GTGTTCTCGTGCTCAGTGATGCACGAGGCTCTGCATAAC |
| | | CACTATACGCAGAAATCATTGTCGCTCAGCCCCGGTAAA |
| | | TAG |
| 3 | PSMA x CD3 T-cell | CAGGTGCAGCTCGTGGAATCCGGTGGCGGCTTGGTGAAA |
| | engager polypeptide- | CCTGGCGAGTCACTGAGATTGTCATGCGCTGCATCAGGT |
| | encoding nucleic acid | TTTACATTCAGTGATTACTATATGTACTGGGTACGGCAG |
| | #2 | GCACCTGGAAAATGCCTTGAGTGGGTCGCTATTATCAGT |
| | | GACGGCGGGTATTATACCTATTACTCCGATATCATTAAA |
| | | GGAAGGTTTACCATCAGCCGAGATAACGCCAAAAACTC |
| | | CCTTTACCTTCAGATGAACAGTCTTAAAGCTGAGGACAC |
| | | TGCTGTTTACTATTGTGCTAGAGGGTTCCCCCTGCTGCGT |
| | | CACGGCGCAATGGATTACTGGGGCCAGGGCACCCTTGTA |
| | | ACCGTAAGCAGTGGTGGGGGGGGTAGTGGTGGTGGTGG |
| | | CTCTGGCGGTGGAGGATCAGACATCCAGATGACCCAAA |
| | | GCCCCTCTAGTCTCAGCGCAAGTGTAGGCGACCGGGTAA |
| | | CCATTACATGTAAGGCATCACAGAATGTGGACACAAATG |
| | | TTGCTTGGTATCAGCAGAAGCCAGGACAGGCTCCTAAGT |
| | | CACTTATCTACAGTGCAAGTTATGTGTATTGGGACGTTC |
| | | CTTCCCGCTTTTCTGGTTCCGCTAGTGGAACTGACTTCAC |
| | | TTTGACTATTTCATCAGTTCAGTCTGAGGACTTCGCCACA |
| | | TATTACTGCCAACAATATGATCAGCAGCTCATTACCTTT |
| | | GGCTGTGGCACAAAGCTGGAAATCAAGTCCGGAGGTGG |
| | | TGGATCCGAGGTGCAGCTGGTCGAGTCTGGAGGAGGATT |
| | | GGTGCAGCCTGGAGGGTCATTGAAACTCTCATGTGCAGC |
| | | CTCTGGATTCACCTTCAATAAGTACGCCATGAACTGGGT |
| | | CCGCCAGGCTCCAGGAAAGGGTTTGGAATGGGTTGCTCG |
| | | CATCAGAAGTAAATATAATAATTATGCAACATATTATGC |
| | | CGATTCAGTGAAAGACAGGTTCACCATCTCCAGAGATGA |
| | | TTCAAAAAACACTGCCTATCTACAAATGAACAACTTGAA |
| | | GACTGAGGACACTGCCGTGTACTACTGTGTGAGACATGG |
| | | GAACTTCGGTAATAGCTACATCTCCTACTGGGCTTACTG |
| | | GGGCCAAGGGACTCTGGTCACCGTGTCCTCAGGTGGTGG |
| | | TGGTTCTGGCGGCGGCGGCTCCGGTGGTGGTGGTTCTCA |
| | | GACTGTTGTGACTCAGGAACCTTCACTCACCGTATCACC |
| | | TGGTGGAACAGTCACACTCACTTGTGGCTCCTCGACTGG |
| | | GGCTGTTACATCTGGCAACTACCCAAACTGGGTCCAACA |
| | | AAAACCAGGTCAGGCACCCCGTGGTCTAATCGGTGGGA |
| | | CTAAGTTCCTCGCCCCCGGTACTCCTGCCAGATTCTCAG |
| | | GCTCCCTGCTTGGAGGCAAGGCTGCCCTCACCCTCTCAG |
| | | GGGTACAGCCAGAGGATGAGGCGGAATATTACTGTGTTC |
| | | TATGGTACAGCAACCGCTGGGTGTTCGGTGGAGGAACCA |
| | | AACTGACTGTCCTAGGCGGTGGTGGAGACAAAACTCAC |
| | | ACATGCCCACCGTGCCCAGCACCTGAACTCCTGGGGGGA |
| | | CCGTCAGTCTTCCTCTTCCCCCCAAAACCCAAGGACACC |
| | | CTCATGATCTCCCGGACCCCTGAGGTCACATGCGTGGTG |
| | | GTGGACGTGAGCCACGAAGACCCTGAGGTCAAGTTCAA |
| | | CTGGTACGTGGACGGCGTGGAGGTGCATAATGCCAAGA |
| | | CAAAGCCGTGCGAGGAGCAGTACGGCAGCACGTACCGT |
| | | TGCGTCAGCGTCCTCACCGTCCTGCACCAGGACTGGCTG |
| | | AATGGCAAGGAGTACAAGTGCAAGGTGTCCAACAAAGC |
| | | CCTCCCAGCCCCCATCGAGAAAACCATCTCCAAAGCCAA |
| | | AGGGCAGCCCCGAGAACCACAGGTGTACACCCTGCCCC |
| | | CATCCCGGGAGGAGATGACCAAGAACCAGGTCAGCCTG |
| | | ACCTGCCTGGTCAAAGGCTTCTATCCCAGCGACATCGCC |
| | | GTGGAGTGGGAGAGCAATGGGCAGCCGGAGAACAACTA |
| | | CAAGACCACGCCTCCCGTGCTGGACTCCGACGGCTCCTT |
| | | CTTCCTCTATAGCAAGCTCACCGTGGACAAGAGCAGGTG |
| | | GCAGCAGGGGAACGTCTTCTCATGCTCCGTGATGCATGA |
| | | GGCTCTGCACAACCACTACACGCAGAAGAGCCTCTCCCT |
| | | GTCTCCGGGTAAAGGTGGAGGAGGTAGCGGAGGAGGCG |
| | | GCTCCGGAGGGGGCGGATCTGGCGGCGGAGGAAGCGGT |
| | | GGGGGCGGCTCCGGAGGGGGCGGCTCTGACAAGACCCA |
| | | CACATGTCCTCCGTGCCCCGCACCCGAGCTGTTGGGAGG |
| | | ACCCTCGGTGTTTTTGTTTCCTCCCAAGCCAAAGGACAC |
| | | GTTGATGATTTCGCGCACTCCAGAGGTCACGTGTGTAGT |
| | | CGTGGACGTGTCACATGAGGACCCCGAGGTAAAGTTCA |
| | | ATTGGTATGTGGACGGAGTCGAGGTCCATAACGCCAAA |
| | | ACAAAGCCGTGCGAAGAACAGTACGGGTCAACGTATAG |
| | | GTGCGTCAGCGTCCTCACTGTGCTGCACCAAGACTGGCT |
| | | CAATGGTAAAGAATACAAGTGCAAGGTGTCGAACAAGG |
| | | CCCTCCCTGCCCCTATCGAGAAAACCATCTCCAAAGCGA |
| | | AGGGGCAGCCGCGAGAACCCCAAGTCTACACGCTGCCG |
| | | CCCTCGCGGGAGGAAATGACCAAAAACCAGGTGTCGCT |
| | | TACGTGTCTTGTGAAAGGGTTCTATCCATCAGATATCGC |
| | | GGTCGAGTGGGAGTCGAACGGCCAGCCCGAAAACAATT |
| | | ACAAAACAACACCTCCGGTCCTCGATTCAGATGGAAGCT |
| | | TCTTCTTGTATTCGAAGCTGACCGTCGATAAGTCAAGGT |
| | | GGCAACAGGGAAATGTGTTCTCGTGCTCAGTGATGCACG |
| | | AGGCTCTGCATAACCACTATACGCAGAAATCATTGTCGC |
| | | TCAGCCCCGGTAAATAG |
| 4 | PSMA x CD3 T-cell | ATGGATATGCGCGTGCCCGCTCAGCTGCTGGGGCTGCTT |
| | engager polypeptide- | CTGCTCTGGCTTCGCGGGGCCCGCTGTCAGGTGCAACTG |
| | encoding nucleic acid | GTGGAAAGCGGTGGCGGCCTTGTGAAGCCTGGGGAATC |
| | #3 | CCTCAGGCTCTCCTGTGCTGCTAGTGGCTTCACGTTCAGC |
| | | GACTACTACATGTATTGGGTGCGGCAGGCTCCAGGAAAG |
| | | TGCCTTGAGTGGGTCGCCATCATTTCCGATGGCGGGTAT |
| | | TACACCTACTACAGCGACATCATCAAGGGTCGGTTTACC |
| | | ATTAGCCGCGACAATGCCAAGAACAGCCTGTATCTGCAG |
| | | ATGAACAGTCTGAAAGCCGAGGATACTGCCGTTTATTAC |
| | | TGTGCAAGAGGGTTTCCCTTGTTGCGTCACGGCGCAATG |
| | | GACTACTGGGGTCAAGGAACACTGGTGACAGTATCATCA |
| | | GGAGGTGGAGGGTCTGGCGGCGGAGGTAGTGGAGGAGG |
| | | TGGGTCCGATATTCAGATGACTCAGTCTCCTTCCTCTCTG |
| | | AGCGCTTCTGTAGGCGATCGAGTCACCATAACCTGCAAA |
| | | GCAAGCCAGAATGTGGACACAAACGTGGCATGGTACCA |
| | | ACAGAAGCCAGGCCAAGCTCCCAAGTCTCTGATCTATTC |
| | | TGCCTCATACGTCTATTGGGATGTTCCCAGTCGGTTTAGT |
| | | GGATCTGCCTCTGGGACAGACTTCACTCTGACTATCTCCT |
| | | CCGTTCAATCCGAGGACTTTGCCACCTACTATTGCCAGC |
| | | AGTATGATCAGCAGCTCATCACATTCGGCTGTGGGACCA |
| | | AACTGGAGATTAAATCCGGAGGTGGAGGGAGCGAAGTG |
| | | CAGCTCGTTGAGAGCGGCGGAGGCCTTGTGCAGCCTGGA |
| | | GGATCCCTGAAGCTGAGCTGTGCCGCATCTGGGTTCACA |
| | | TTCAACAAGTATGCCATGAATTGGGTGCGTCAGGCTCCT |
| | | GGTAAAGGCTTGGAGTGGGTAGCCCGGATTAGGAGCAA |
| | | ATATAATAACTATGCCACCTACTATGCTGACTCTGTCAA |
| | | GGACCGCTTTACAATCTCTCGGGATGACTCCAAGAATAC |
| | | TGCCTACCTCCAGATGAACAACCTCAAGACGGAGGATAC |
| | | CGCCGTCTACTATTGCGTCCGACATGGCAACTTTGGGAA |
| | | CTCCTACATCTCCTATTGGGCTTATTGGGGCCAGGGGAC |
| | | TCTTGTCACAGTCTCATCCGGCGGTGGGGGCTCTGGTGG |
| | | AGGTGGGTCAGGAGGTGGTGGGAGCCAAACCGTTGTAA |
| | | CACAGGAGCCCTCCTTGACTGTGTCACCTGGCGGAACGG |
| | | TGACGCTGACTTGTGGCTCATCAACTGGAGCCGTAACAT |
| | | CTGGAAATTACCCTAACTGGGTGCAGCAGAAGCCTGGTC |
| | | AGGCACCCAGAGGACTGATAGGCGGAACCAAATTTCTC |
| | | GCTCCAGGTACTCCAGCCCGGTTTTCCGGGTCCTTGCTG |
| | | GGCGGTAAGGCTGCCCTGACACTGAGCGGAGTTCAGCC |
| | | AGAGGATGAAGCAGAATACTATTGCGTGCTGTGGTACTC |
| | | CAACAGGTGGGTATTTGGAGGCGGCACCAAGCTCACGG |
| | | TCCTCGGCGGAGGTGGAGACAAAACACACACTTGTCCTC |
| | | CCTGTCCTGCACCCGAACTTCTGGGAGGCCCCTCTGTGTT |
| | | CCTTTTCCCACCCAAGCCCAAAGATACCTTGATGATCAG |
| | | CCGTACGCCAGAGGTTACATGCGTGGTCGTTGACGTGTC |
| | | TCATGAAGATCCCGAAGTCAAGTTCAACTGGTACGTGGA |
| | | TGGGGTGGAGGTACATAATGCAAAGACCAAGCCCTGTG |
| | | AGGAGCAGTATGGGTCAACCTATCGGTGTGTGTCTGTGC |
| | | TGACCGTGCTGCATCAAGATTGGTTGAATGGGAAGGAAT |
| | | ATAAGTGCAAGGTGTCAAATAAGGCTCTGCCCGCACCCA |
| | | TTGAAAAGACCATAAGTAAGGCCAAGGGGCAGCCTAGG |
| | | GAACCCCAGGTATACACTCTGCCTCCATCCCGTGAGGAG |
| | | ATGACAAAGAATCAAGTGAGCCTCACCTGTCTGGTTAAA |
| | | GGCTTCTACCCCAGTGATATCGCTGTGGAGTGGGAGTCA |
| | | AACGGGCAGCCCGAAAACAACTACAAAACCACACCTCC |
| | | AGTCTTGGATAGCGACGGATCCTTCTTTCTGTACTCTAAA |
| | | CTCACCGTGGACAAAAGTAGATGGCAGCAAGGCAACGT |
| | | TTTCAGTTGCTCTGTGATGCACGAAGCTCTCCATAATCAT |
| | | TATACTCAGAAGAGCCTGTCCCTTTCTCCTGGAAAAGGG |
| | | GGGGGTGGCTCCGGCGGTGGCGGCAGTGGAGGCGGAGG |
| | | CTCTGGTGGTGGCGGTTCCGGTGGCGGGGGAAGCGGCG |
| | | GCGGTGGGAGTGACAAGACACATACCTGTCCTCCATGCC |
| | | CAGCTCCCGAACTGTTGGGCGGACCCAGCGTGTTTCTGT |
| | | TTCCACCAAAGCCTAAAGACACCCTGATGATTTCAAGGA |
| | | CTCCTGAGGTGACATGCGTGGTAGTGGATGTCAGCCACG |
| | | AGGACCCCGAAGTGAAATTCAATTGGTATGTAGACGGG |
| | | GTGGAGGTCCACAATGCCAAGACTAAACCCTGCGAGGA |
| | | GCAGTACGGGTCCACTTACCGATGCGTTAGTGTTCTCAC |
| | | AGTCCTTCACCAGGACTGGCTGAACGGAAAGGAGTACA |
| | | AATGCAAAGTGTCCAACAAAGCCCTGCCAGCCCCAATCG |
| | | AGAAAACAATCAGCAAGGCCAAGGGTCAACCTCGAGAA |
| | | CCCCAAGTGTATACTCTGCCACCCTCTCGCGAAGAGATG |
| | | ACCAAGAATCAGGTTTCCCTCACCTGTTTGGTCAAAGGC |
| | | TTTTACCCCTCTGACATTGCTGTTGAATGGGAGAGTAAC |
| | | GGCCAGCCTGAGAACAACTATAAAACCACCCCACCAGT |
| | | GCTTGATAGTGATGGCTCCTTCTTCCTGTACAGCAAGCT |
| | | GACAGTGGACAAATCTCGCTGGCAACAGGGCAATGTCTT |
| | | TAGTTGCTCCGTCATGCACGAGGCACTGCACAATCACTA |
| | | CACTCAGAAGTCACTGAGTCTGAGCCCTGGGAAGTAG |
| 5 | PSMA x CD3 T-cell | CAGGTGCAACTGGTGGAAAGCGGTGGCGGCCTTGTGAA |
| | engager polypeptide- | GCCTGGGGAATCCCTCAGGCTCTCCTGTGCTGCTAGTGG |
| | encoding nucleic acid | CTTCACGTTCAGCGACTACTACATGTATTGGGTGCGGCA |
| | #4 | GGCTCCAGGAAAGTGCCTTGAGTGGGTCGCCATCATTTC |
| | | CGATGGCGGGTATTACACCTACTACAGCGACATCATCAA |
| | | GGGTCGGTTTACCATTAGCCGCGACAATGCCAAGAACAG |
| | | CCTGTATCTGCAGATGAACAGTCTGAAAGCCGAGGATAC |
| | | TGCCGTTTATTACTGTGCAAGAGGGTTTCCCTTGTTGCGT |
| | | CACGGCGCAATGGACTACTGGGGTCAAGGAACACTGGT |
| | | GACAGTATCATCAGGAGGTGGAGGGTCTGGCGGCGGAG |
| | | GTAGTGGAGGAGGTGGGTCCGATATTCAGATGACTCAGT |
| | | CTCCTTCCTCTCTGAGCGCTTCTGTAGGCGATCGAGTCAC |
| | | CATAACCTGCAAAGCAAGCCAGAATGTGGACACAAACG |
| | | TGGCATGGTACCAACAGAAGCCAGGCCAAGCTCCCAAG |
| | | TCTCTGATCTATTCTGCCTCATACGTCTATTGGGATGTTC |
| | | CCAGTCGGTTTAGTGGATCTGCCTCTGGGACAGACTTCA |
| | | CTCTGACTATCTCCTCCGTTCAATCCGAGGACTTTGCCAC |
| | | CTACTATTGCCAGCAGTATGATCAGCAGCTCATCACATT |
| | | CGGCTGTGGGACCAAACTGGAGATTAAATCCGGAGGTG |
| | | GAGGGAGCGAAGTGCAGCTCGTTGAGAGCGGCGGAGGC |
| | | CTTGTGCAGCCTGGAGGATCCCTGAAGCTGAGCTGTGCC |
| | | GCATCTGGGTTCACATTCAACAAGTATGCCATGAATTGG |
| | | GTGCGTCAGGCTCCTGGTAAAGGCTTGGAGTGGGTAGCC |
| | | CGGATTAGGAGCAAATATAATAACTATGCCACCTACTAT |
| | | GCTGACTCTGTCAAGGACCGCTTTACAATCTCTCGGGAT |
| | | GACTCCAAGAATACTGCCTACCTCCAGATGAACAACCTC |
| | | AAGACGGAGGATACCGCCGTCTACTATTGCGTCCGACAT |
| | | GGCAACTTTGGGAACTCCTACATCTCCTATTGGGCTTATT |
| | | GGGGCCAGGGGACTCTTGTCACAGTCTCATCCGGCGGTG |
| | | GGGGCTCTGGTGGAGGTGGGTCAGGAGGTGGTGGGAGC |
| | | CAAACCGTTGTAACACAGGAGCCCTCCTTGACTGTGTCA |
| | | CCTGGCGGAACGGTGACGCTGACTTGTGGCTCATCAACT |
| | | GGAGCCGTAACATCTGGAAATTACCCTAACTGGGTGCAG |
| | | CAGAAGCCTGGTCAGGCACCCAGAGGACTGATAGGCGG |
| | | AACCAAATTTCTCGCTCCAGGTACTCCAGCCCGGTTTTCC |
| | | GGGTCCTTGCTGGGCGGTAAGGCTGCCCTGACACTGAGC |
| | | GGAGTTCAGCCAGAGGATGAAGCAGAATACTATTGCGT |
| | | GCTGTGGTACTCCAACAGGTGGGTATTTGGAGGCGGCAC |
| | | CAAGCTCACGGTCCTCGGCGGAGGTGGAGACAAAACAC |
| | | ACACTTGTCCTCCCTGTCCTGCACCCGAACTTCTGGGAG |
| | | GCCCCTCTGTGTTCCTTTTCCCACCCAAGCCCAAAGATAC |
| | | CTTGATGATCAGCCGTACGCCAGAGGTTACATGCGTGGT |
| | | CGTTGACGTGTCTCATGAAGATCCCGAAGTCAAGTTCAA |
| | | CTGGTACGTGGATGGGGTGGAGGTACATAATGCAAAGA |
| | | CCAAGCCCTGTGAGGAGCAGTATGGGTCAACCTATCGGT |
| | | GTGTGTCTGTGCTGACCGTGCTGCATCAAGATTGGTTGA |
| | | ATGGGAAGGAATATAAGTGCAAGGTGTCAAATAAGGCT |
| | | CTGCCCGCACCCATTGAAAAGACCATAAGTAAGGCCAA |
| | | GGGGCAGCCTAGGGAACCCCAGGTATACACTCTGCCTCC |
| | | ATCCCGTGAGGAGATGACAAAGAATCAAGTGAGCCTCA |
| | | CCTGTCTGGTTAAAGGCTTCTACCCCAGTGATATCGCTGT |
| | | GGAGTGGGAGTCAAACGGGCAGCCCGAAAACAACTACA |
| | | AAACCACACCTCCAGTCTTGGATAGCGACGGATCCTTCT |
| | | TTCTGTACTCTAAACTCACCGTGGACAAAAGTAGATGGC |
| | | AGCAAGGCAACGTTTTCAGTTGCTCTGTGATGCACGAAG |
| | | CTCTCCATAATCATTATACTCAGAAGAGCCTGTCCCTTTC |
| | | TCCTGGAAAAGGGGGGGGTGGCTCCGGCGGTGGCGGCA |
| | | GTGGAGGCGGAGGCTCTGGTGGTGGCGGTTCCGGTGGC |
| | | GGGGGAAGCGGCGGCGGTGGGAGTGACAAGACACATAC |
| | | CTGTCCTCCATGCCCAGCTCCCGAACTGTTGGGCGGACC |
| | | CAGCGTGTTTCTGTTTCCACCAAAGCCTAAAGACACCCT |
| | | GATGATTTCAAGGACTCCTGAGGTGACATGCGTGGTAGT |
| | | GGATGTCAGCCACGAGGACCCCGAAGTGAAATTCAATT |
| | | GGTATGTAGACGGGGTGGAGGTCCACAATGCCAAGACT |
| | | AAACCCTGCGAGGAGCAGTACGGGTCCACTTACCGATGC |
| | | GTTAGTGTTCTCACAGTCCTTCACCAGGACTGGCTGAAC |
| | | GGAAAGGAGTACAAATGCAAAGTGTCCAACAAAGCCCT |
| | | GCCAGCCCCAATCGAGAAAACAATCAGCAAGGCCAAGG |
| | | GTCAACCTCGAGAACCCCAAGTGTATACTCTGCCACCCT |
| | | CTCGCGAAGAGATGACCAAGAATCAGGTTTCCCTCACCT |
| | | GTTTGGTCAAAGGCTTTTACCCCTCTGACATTGCTGTTGA |
| | | ATGGGAGAGTAACGGCCAGCCTGAGAACAACTATAAAA |
| | | CCACCCCACCAGTGCTTGATAGTGATGGCTCCTTCTTCCT |
| | | GTACAGCAAGCTGACAGTGGACAAATCTCGCTGGCAAC |
| | | AGGGCAATGTCTTTAGTTGCTCCGTCATGCACGAGGCAC |
| | | TGCACAATCACTACACTCAGAAGTCACTGAGTCTGAGCC |
| | | CTGGGAAGTAG |
| 6 | Signal peptide amino | MDMRVPAQLLGLLLLWLRGARC |
| | acid sequence #1 | |
| 7 | Signal peptide amino | MAWALLLLTLLTQGTGSWA |
| | acid sequence #2 | |
| 8 | Signal peptide amino | MTCSPLLLTLLIHCTGSWA |
| | acid sequence #3 | |
| 9 | Signal peptide amino | MEWTWRVLFLVAAATGAHS |
| | acid sequence #4 | |
| 10 | Signal peptide amino | MEWSWVFLFFLSVTTGVHS |
| | acid sequence #5 | |
| 11 | Signal peptide amino | MDIRAPTQLLGLLLLWLPGAKC |
| | acid sequence #6 | |
| 12 | Signal peptide amino | MDIRAPTQLLGLLLLWLPGARC |
| | acid sequence #7 | |
| 13 | Signal peptide amino | MDMRAPTQLLGLLLLWLPGARC |
| | acid sequence #8 | |
| 14 | Signal peptide amino | MDTRAPTQLLGLLLLWLPGATF |
| | acid sequence #9 | |
| 15 | Signal peptide amino | MDTRAPTQLLGLLLLWLPGARC |
| | acid sequence #10 | |
| 16 | Signal peptide amino | METGLRWLLLVAVLKGVQC |
| | acid sequence #11 | |
| 17 | Signal peptide amino | METGLRWLLLVAVLKGVQCQE |
| | acid sequence #12 | |
| 18 | Signal peptide amino | MEAPAQLLFLLLLWLPDTTG |
| | acid sequence #13 | |
| 19 | Signal peptide amino | METPAQLLFLLLLWLPDTTG |
| | acid sequence #14 | |
| 20 | Optimized signal | ATGGATATGCGCGTGCCCGCTCAGCTGCTGGGGCTGCTT |
| | peptide nucleic acid | CTGCTCTGGCTTCGCGGGGCCCGCTGT |
| | sequence #1 | |
| 21 | Optimized signal | ATGGCCTGGGCTCTGCTGCTCCTCACCCTCCTCACTCAGG |
| | peptide nucleic acid | GGACAGGGTCCTGGGCC |
| | sequence #2 | |
| 22 | Optimized signal | ATGGCCTGGGCTCTGCTGCTCCTCACCCTCCTCACTCAGG |
| | peptide nucleic acid | GGACCGGGTCCTGGGCC |
| | sequence #3 | |
| 23 | Splice variant isoform | EDFATYYCQQYDQQLITFGCGTKLEIKSGGGGSEVQLVESG |
| | of PSMA x CD3 T-cell | GGLVQPGGSLKLSCAASGFTFNKYAMNWVRQAPGKGLEW |
| | polypeptide amino acid | VARIRSKYNNYATYYADSVKDRFTISRDDSKNTAYLQMNN |
| | sequence | LKTEDTAVYYCVRHGNFGNSYISYWAYWGQGTLVTVSSG |
| | | GGGSGGGGSGGGGSQTVVTQEPSLTVSPGGTVTLTCGSST |
| | | GAVTSGNYPNWVQQKPGQAPRGLIGGTKFLAPGTPARFSG |
| | | SLLGGKAALTLSGVQPEDEAEYYCVLWYSNRWVFGGGTK |
| | | LTVLGGGGDKTHTCPPCPAPELLGGPSVFLFPPKPKDTLMIS |
| | | RTPEVTCVVVDVSHEDPEVKFNWYVDGVEVHNAKTKPCE |
| | | EQYGSTYRCVSVLTVLHQDWLNGKEYKCKVSNKALPAPIE |
| | | KTISKAKGQPREPQVYTLPPSREEMTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGKGGGGSGG |
| | | GGSGGGGSGGGGSGGGGSGGGGSDKTHTCPPCPAPELLGG |
| | | PSVFLFPPKPKDTLMISRTPEVTCVVVDVSHEDPEVKFNWY |
| | | VDGVEVHNAKTKPCEEQYGSTYRCVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPG |

What is claimed is:

1. A method for producing a recombinant protein composition comprising a reduced amount of low molecular weight (LMW) species of the protein, the method comprising:
culturing a mammalian cell expressing a nucleic acid encoding the protein in a cell culture medium for a period of time during which the protein is expressed and secreted by the mammalian cell, wherein the pH of the culture medium is maintained at about 6.90 or less; and
recovering the expressed protein from the cell culture medium to obtain the recombinant protein composition, wherein the composition comprises less than 20% total LMW species of the protein, and wherein the protein comprises the amino acid sequence of SEQ ID NO: 1.

2. The method of claim 1, wherein the culture medium is maintained at a pH from about 6.70 to about 6.90.

3. The method of claim 1, wherein the culture medium is maintained at a pH of about 6.80.

4. The method of any one of claims 1 to 3, wherein the period of time is at least 3 days.

5. The method of any one of claims 1 to 4, wherein the period of time is about 12 days to about 15 days.

6. The method of any one of claims 1 to 5, wherein the mammalian cell is cultured in a perfusion culture.

7. The method of any one of claims 1 to 6, wherein the mammalian cell is cultured to a viable cell density between 300×10⁵ cells/mL and 800×10⁵ cells/mL.

8. The method of any one of claims 1 to 7, wherein the expressed protein is recovered from the cell culture medium by microfiltration.

9. The method of any one of claims 1 to 8, wherein the mammalian cell is a CHO cell.

10. The method of any one of claims 1 to 9, wherein the nucleic acid encoding the protein comprises the nucleotide sequence of SEQ ID NO: 2 or SEQ ID NO: 3.

11. The method of any one of claims 1 to 9, wherein the nucleic acid encoding the protein comprises the nucleotide sequence of SEQ ID NO: 4 or SEQ ID NO: 5.

12. The method of any one of claims 1 to 11, wherein the composition is harvested cell culture fluid.

13. The method of any one of claims 1 to 12, wherein the composition comprises about 15% or less total LMW species of the protein.

14. The method of any one of claims 1 to 12, wherein the composition comprises about 10% or less total LMW species of the protein.

15. The method of any one of claims 1 to 12, wherein the composition comprises about 2% to about 10% total LMW species of the protein.

16. The method of any one of claims 1 to 15, wherein the LMW species comprises a splice variant isoform of the protein.

17. The method of any one of claims 1 to 16, wherein the amount of LMW species in the composition is determined by a reduced capillary electrophoresis-sodium dodecyl sulfate method.

18. A method for reducing expression and secretion of alternative splice variant isoforms of a recombinant protein from a mammalian cell, the method comprising:
transfecting a mammalian cell with a nucleic acid comprising a first polynucleotide encoding a signal peptide and a second polynucleotide encoding the recombinant protein, wherein the first polynucleotide is in the same open reading frame as the second polynucleotide, wherein the first polynucleotide comprises a GGG codon encoding glycine for any glycine residue occurring within the six carboxy-terminal amino acids of the signal peptide;
culturing the mammalian cell in a cell culture medium under conditions where the recombinant protein is expressed and secreted into the medium; and
recovering the recombinant protein from the cell culture medium to obtain a recombinant protein composition.

19. The method of claim 18, wherein the first polynucleotide encodes a signal peptide comprising the amino acid sequence of any one of SEQ ID NOs: 6-19.

20. The method of claim 19, wherein the first polynucleotide encodes a signal peptide comprising the amino acid sequence of SEQ ID NO: 6.

21. The method of claim 18, wherein the first polynucleotide comprises the nucleotide sequence of SEQ ID NO: 20.

22. The method of any one of claims 18 to 21, wherein the recombinant protein is a single chain T-cell engaging molecule.

23. The method of claim 22, wherein the recombinant protein comprises the amino acid sequence of SEQ ID NO: 1.

24. The method of claim 23, wherein the second polynucleotide comprises the nucleotide sequence of SEQ ID NO: 5.

25. The method of any one of claims 18 to 21, wherein the recombinant protein is an antibody or binding fragment thereof.

26. The method of claim 18, wherein the nucleic acid comprises the nucleotide sequence of SEQ ID NO: 4.

27. The method of any one of claims 18 to 26, wherein the mammalian cell is a CHO cell.

28. The method of any one of claims 18 to 27, wherein the recombinant protein composition comprises about 10% or less total LMW species of the protein.

29. The method of any one of claims 18 to 28, wherein the culture medium is maintained at a pH of about 6.90 or less.

30. The method of claim 29, wherein the culture medium is maintained at a pH of about 6.70 to about 6.90.

31. The method of claim 29, wherein the culture medium is maintained at a pH of about 6.80.

32. An isolated nucleic acid encoding a single chain PSMA×CD3 T-cell engaging molecule comprising a nucleotide sequence selected from SEQ ID NO: 2, SEQ ID NO: 3, SEQ ID NO: 4, and SEQ ID NO: 5.

33. An expression vector comprising the isolated nucleic acid of claim 32.

34. A mammalian host cell transformed with the isolated nucleic acid of claim 32.

35. A mammalian host cell transformed with the expression vector of claim 33.

36. The mammalian host cell of claim 34 or 35, wherein the host cell is a CHO cell.

37. A method of producing a single chain PSMA×CD3 T-cell engaging molecule comprising:
culturing the mammalian host cell of any one of claims 34 to 36 in a cell culture medium under conditions where the T-cell engaging molecule is expressed; and
recovering the T-cell engaging molecule from the culture medium or host cell.

38. A recombinant protein composition produced by the method of claim 1 or claim 18.

39. A composition comprising a single chain PSMA×CD3 T-cell engaging molecule and one or more LMW species thereof, wherein the composition comprises less than 20% total LMW species of the T-cell engaging molecule, and wherein the T-cell engaging molecule comprises the amino acid sequence of SEQ ID NO: 1.

40. The composition of claim 39, wherein the composition comprises about 15% or less total LMW species of the T-cell engaging molecule.

41. The composition of claim 39, wherein the composition comprises about 10% or less total LMW species of the T-cell engaging molecule.

42. The composition of claim 39, wherein the composition comprises about 2% to about 10% total LMW species of the T-cell engaging molecule.

43. The composition of claim 39, wherein the composition comprises about 2% to about 6% total LMW species of the T-cell engaging molecule.

44. The composition of any one of claims 39 to 43, wherein the LMW species comprises a splice variant isoform of the T-cell engaging molecule.

45. The composition of any one of claims 39 to 44, wherein the amount of LMW species in the composition is determined by a reduced capillary electrophoresis-sodium dodecyl sulfate method.

46. A pharmaceutical formulation comprising the composition of any one of claims 38 to 45 and one or more pharmaceutically acceptable excipients.

47. A method for treating a PSMA-expressing cancer in a patient in need thereof comprising administering to the patient the pharmaceutical formulation of claim 46.

48. The method of claim 47, wherein the PSMA-expressing cancer is prostate cancer.

49. A composition according to any one of claims 38 to 45 for use in a method for treating a PSMA-expressing cancer in a patient in need thereof.

50. The composition for use according to claim 49, wherein the PSMA-expressing cancer is prostate cancer.

51. Use of a composition according to any one of claims 38 to 45 in the preparation of a medicament for treating a PSMA-expressing cancer in a patient in need thereof.

52. The use of claim 51, wherein the PSMA-expressing cancer is prostate cancer.
